(19) United States
(12) Patent Application Publication
(10) Pub. No.: US 2009/0181115 A1
(43) Pub. Date: Jul. 16, 2009

(54) Method of Screening for Compounds That Alter Skin and/or Hair Pigmentation

(76) Inventors: Wendy Filsell, Sharnbrook (GB); Rebecca Susan Ginger, Sharnbrook (GB); Martin Richard Green, Sharnbrook (GB); Carl Dudley Jarman, Sharnbrook (GB); Richard Martin Ogborne, Sharnbrook (GB); Paul Petrus Marius Schnetkamp, Calgary (CA); Stephen Wilson, Sharnbrook (GB)

Correspondence Address: MORGAN LEWIS & BOCKIUS LLP, 1111 PENNSYLVANIA AVENUE NW, WASHINGTON, DC, 20004, US

(21) Appl. No.: 12/301,839

(22) PCT Filed: May 30, 2007
(86) PCT No.: PCT/EP07/55224

§ 371 Date: Feb. 2, 2009

(30) Foreign Application Priority Data
May 31, 2006 (EP) 06114771.6

(51) Int. Cl. A61K 36/23; C12N 15/12; C12N 15/63; C12N 5/10; C07K 14/435; A61P 17/00

(52) U.S. Cl. 424/776; 536/23.4; 435/320.1; 435/348; 435/369; 530/300

(57) ABSTRACT

The invention provides a method of identifying compounds that either increase or decrease skin and/or hair pigmentation, the method comprising determining the ability of a test compound to modulate NCKX-mediated calcium ion movement across a membrane (e.g. NCKX5). The method may comprise the steps of exposing a membrane comprising a NCKX molecule or variant, fusion or derivative thereof to a test compound and measuring either directly or indirectly the calcium ion concentration on one or both sides of the membrane. The invention also relates to kits, nucleic acid molecules, polypeptides and cells useful in the method of the invention.

The invention relates to methods of identifying compounds having activity in altering skin pigmentation and nucleic acid molecules, polypeptides and cells useful in said methods.

The skin is the largest organ in the body and has roles in thermoregulation, protection from physical and chemical injury, protection from infection and manufacture of Vitamin D. There is a broad range of skin colours which can be correlated to climates, continents and cultures. Predominantly darker skins are located in hotter climates closer to the equator and are thought to provide protection against UV radiation and the heat. Lighter skins are found in cooler areas where there is less need for UV protection and are also associated with increased vitamin D production

The principal pigments responsible for skin colour are carotene, haemoglobin and in particular melanin. Melanin is composed of two major sub-types, the darker eumelanin and lighter pheomelanin. Melanin is synthesised by melanocytes, and combined with other proteins into granules which are then redistributed to keratinocytes. The amount of melanin is influenced by exposure to UV radiation (tanning) and a darker skin can therefore be achieved by increasing the amount of melanin in the skin.

The genetic basis for constitutive and induced skin and hair pigmentation is not yet fully understood and it is hypothesised that a plurality of genes are involved in pigmentation. Different variants of these genes influence the skin colour phenotype of an individual before external factors such as sunlight influence skin colour.

### SUMMARY OF THE INVENTION

In a first aspect of the invention there is provided a method of identifying compounds that either increase or decrease skin and/or hair pigmentation or change the melanin composition of skin and/or hair, the method comprising determining the ability of a test compound to modulate NCKX-mediated calcium ion movement across a membrane.

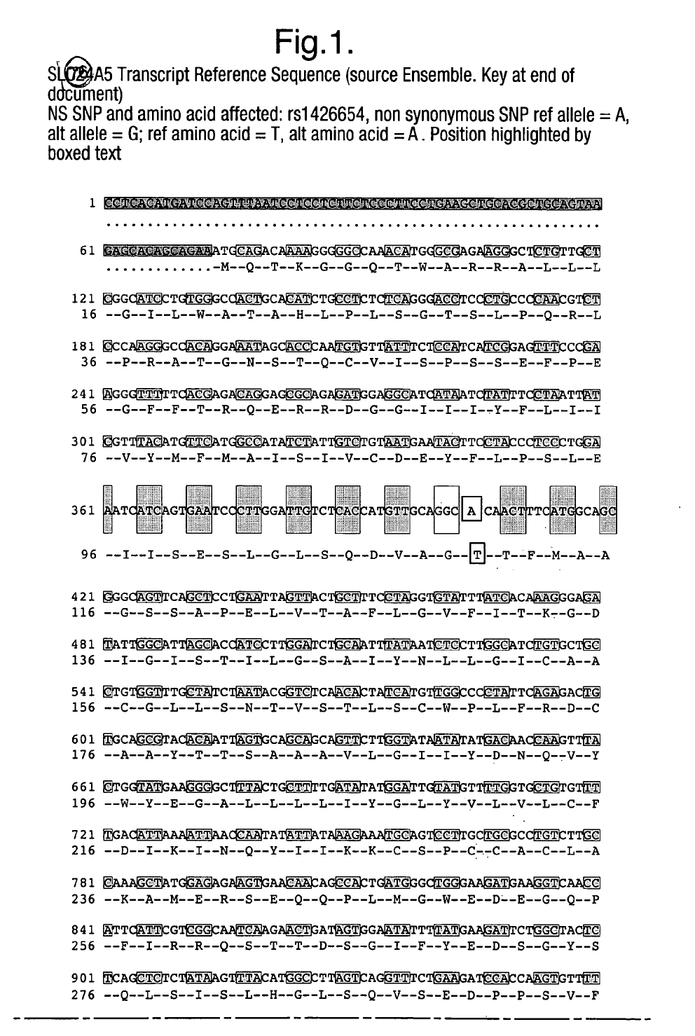

The NCKX molecule may be derived from any species, in particular mammals and most preferably humans. Examples of NCKX molecules are NCKX1 (gene accession no. NM_{—}004727, protein accession no. -NP_{—}004718); NCKX2 gene accession no. NM_{—}020344, protein accession no. -NP_{—}065077); NCKX3 (gene accession no. NM_{—}020689, protein accession no. NP_{—}065740); NCKX4 (gene accession nos. NM_{—}153648, NM_{—}153646, NM_{—}153647; protein accession nos. NP_{—}705934, NP_{—}705932, NP_{—}705933); NCKX5 (see FIG. 1 for sequence and gene accession nos. NM_{—}205850 XM_{—}208771, protein accession nos. NP_{—}995322 XP_{—}208771) and NCKX6 (gene accession no. NM_{—}024959, protein accession no. NP_{—}079235). (See Cai & Lytton (2004) Mol Biol & Evolution vol 21 no 9 pg 1692-1703 and Schnetkamp (2004) Pflugers Arch—Eur J Physiol vol 447 pg 683-688)

Preferably the NCKX molecule is NCKX5. NCKX5 is also known as SLC24A5.

Preferably the method comprises the steps of exposing a membrane comprising a NCKX molecule or variant, fusion or derivative thereof to a test compound and measuring either directly or indirectly the calcium ion concentration on one or both sides of the membrane.

The method of the invention may comprise the steps of:
(a) providing a membrane comprising at least one NCKX molecule or functionally equivalent variants, fusions or derivatives thereof, wherein said membrane separates two distinct compartments;
(b) measuring the Calcium ion (Ca²⁺) concentration in one or both compartments before exposure to one or more test compounds;
(c) exposing the membrane to one or more test compounds;
(d) measuring the Calcium ion (Ca²⁺) concentration in one or both compartments after exposure to one or more test compounds;
(e) identifying the amount of Calcium ion (Ca²⁺) movement across the membrane by comparing the concentrations measured in step (b) and step (d);

The method of the invention may also further comprise comparing the calcium movement in response to a test compound with a control value. Such a comparison may be achieved using the following steps:
(f) repeating the above steps (a), (b), (d) and (e) to provide a control result for the change in the Calcium ion (Ca²⁺) concentration without exposure to one or more test compounds;
(g) comparing the amount of Calcium ion (Ca²⁺) movement across the membrane identified in step (e) after exposure to the test compound, amount of Calcium ion (Ca²⁺) movement across the membrane in the control of step (f)
(h) identifying whether the amount of Calcium ion (Ca²⁺) movement across the membrane has increased, decreased or stayed the same in response to exposure to the test compound(s).

An alternative control method would be to compare the amount of Calcium ion movement across a control membrane which does not contain a NCKX protein.

NCKX5 (also known as SLC24A5) is a member of the Sodium, Calcium/Potassium exchanger family (NCKX) and has been found to influence pigmentation in Zebrafish. In the Zebrafish a mutation in NCKX5 exhibits a reduction in pigmentation associated with the “golden” mutation (Lamason (2005) Science 310 pp 1782-1786).

Studies of other Sodium-Calcium exchangers have been conducted and shown that NCKX1 and NCKX2, for example, exhibit calcium exchange functions in human photoreceptors, bovine heart muscle (Winkfein (2003) Biochemistry 42 pp 543-552 and Schnetkamp (1996) Biochem. Cell. Biol. 74 pp 535-539) and arterial smooth muscle Epublication: Doug (2006) American Journal Physiol. Heart Circ. Physiol (Apr. 14^{th} 2006) doi:10.1152/ajpheart.00196.2006.

The applicant has now shown that NCKX5 is associated with sodium-potassium/calcium exchange function in melanocytes and that this function is closely correlated to skin pigmentation. In addition, the applicant has shown that NCKX5 exists in two allelic forms at amino acid 111 due to a single nucleotide polymorphism (SNP). The Ala111 version is correlated with increased calcium movement and is found predominantly in dark skin. The Thr111 version is closely correlated to decreased calcium movement and is found predominantly in lighter skin. (See examples 1 and 2).

The sequence of NCKX5 including the position of the SNP is shown in FIG. 1.

Preferably, an increase in the amount of Calcium ion (Ca²⁺ movement across the membrane indicates the test compound(s) increase skin pigmentation and a decrease in the amount of Calcium ion (Ca²⁺) movement across the membrane indicates the test compound(s) decrease skin pigmentation.

Optionally, the method of the invention further comprises the step of:
(i) isolating the one or more test compounds.

Further optionally, the method comprises the step of:
(j) formulating the one or more test compounds isolated in step (i) into a cosmetic or pharmaceutical formulation.

Conveniently, the membrane is a biological membrane, such as a cell membrane that is preferably part of an intact cell.

Preferred sources of cell membranes and/or intact cells are Hamster Embryonic Kidney (HEK) cells, High five insect cells, yeast cells, dictyostelium cells, tobacco plant cells, p53 deficient cell line H1299 and/or bacteria.

Advantageously, the NCKX5 molecule is located in the membrane naturally or is artificially targeted to the membrane or is reconstituted into an artificial membrane.

Preferably when the NCKX5 is artificially targeted to the membrane it is done by linking a leader sequence and/or tag that targets polypeptides to and for inclusion in a membrane or by deletion of internal compartment retention signals, such as ArgArg motifs.

Conveniently, the leader sequence is derived from NCKX2 or 4, yeast α mating factor, NCX proteins, TGFbeta, haemagglutinin or viral surface proteins.

Preferably the leader sequence is the N terminal sequence of hsNCKX2 (amino acids 1 to 120).

Advantageously, the calcium ion (Ca²⁺) concentration and/or movement is measured using a method selected from Ca²⁺ sensitive dyes (fluorescent and/or non-fluorescent), electrophysiological methods (e.g. patch clamp), radioactive Calcium (⁴⁵Ca²⁺) or conventional mass spectrometry.

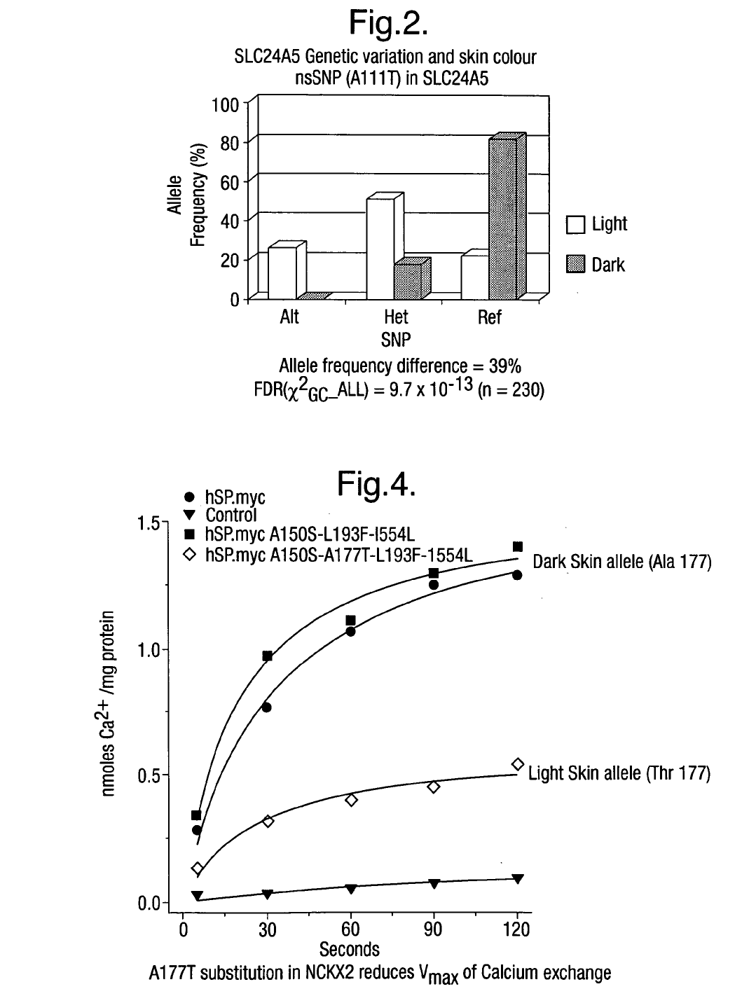

Preferably, the NCKX5 molecule or functionally equivalent variant, fusion or derivative thereof possesses a single nucleotide polymorphism (SNP) at the codon for amino acid residue 111. The SNP at the codon for amino acid residue 111 can code for either Alanine or Threonine (DVAGA/TTFMAAG) (see FIG. 3).

The method may also include the step of testing the selectivity of the test compound (i.e. does it affect any other ion channels) by using broadly known techniques such as electrophysiological methods such as patch clamping.

In a second aspect of the invention there is a nucleic acid molecule encoding a fusion protein comprising the nucleic acid molecule encoding NCKX molecule or a functionally equivalent variant, fusion or derivative thereof and a nucleic acid molecule encoding a membrane targeting leader peptide and/or tag.

The chimeric nucleic acid molecule may encode a chimera comprising the N terminus of NCK2 and NCKX5 from 151 to end; or N-terminus 1-200 amino acids of NCKX4 and NCKX5 from 151 to end. Alternatively the cimera may include the C terminus of NCKX2 (11 amino acids) instead of the final 11 amino acids of NCKX5.

Preferably, the membrane targeting leader peptide and/or tag is derived from NCKX2 or 4, yeast α mating factor, NCX proteins, TGFbeta, haemagglutinin or viral surface proteins.

Most preferably the leader sequence is the N terminal sequence of hsNCKX2 (amino acids 1 to 120).

Advantageously, the nucleic acid molecule encoding the NCKX molecule or a functionally equivalent variant, fusion or derivative thereof includes a single nucleotide polymorphism (SNP) at the codon for amino acid residue 111. The SNP at amino acid residue 111 can code for either Alanine or Threonine.

Thus, the isolated nucleic acid molecule is suitable for expressing a polypeptide of the invention. By ‘suitable for expressing’ is meant that the nucleic acid molecule is a polynucleotide that may be translated to form the polypeptide, for example RNA, or that the polynucleotide (which is preferably DNA) encoding the polypeptide of the invention is inserted into an expression vector, such as a plasmid, in proper orientation and correct reading frame for expression. The polynucleotide may be linked to the appropriate transcriptional and translational regulatory control nucleotide sequences recognised by any desired host; such controls may be incorporated in the expression vector.

The nucleic acid molecule of the invention may be DNA or RNA, preferably DNA.

The DNA is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker, all of which are incorporated herein by reference.

Hence, in a third aspect of the invention there is provided an expression vector comprising a nucleic acid molecule of the second aspect of the invention.

The DNA encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

The DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Bacterial promoters include the E. coli lacI and lacZ promoters, the T3 and T7 promoters, the gpt promoter, the phage λ PR and PL promoters, the phoA promoter and the trp promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome binding site for translation. (Hastings et al, International Patent No. WO 98/16643, published 23 Apr. 1998)

Many expression systems are known, including systems employing: bacteria (e.g. E. coli and Bacillus subtilis) transformed with, for example, recombinant bacteriophage, plasmid or cosmid DNA expression vectors; yeasts (e.g. Saccaromyces cerevisiae) transformed with, for example, yeast expression vectors; insect cell systems transformed with, for example, viral expression vectors (e.g. baculovirus); plant cell systems transfected with, for example viral or bacterial expression vectors; animal cell systems transfected with, for example, adenovirus expression vectors.

The vectors can include a prokaryotic replicon, such as the Col E1 ori, for propagation in a prokaryote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

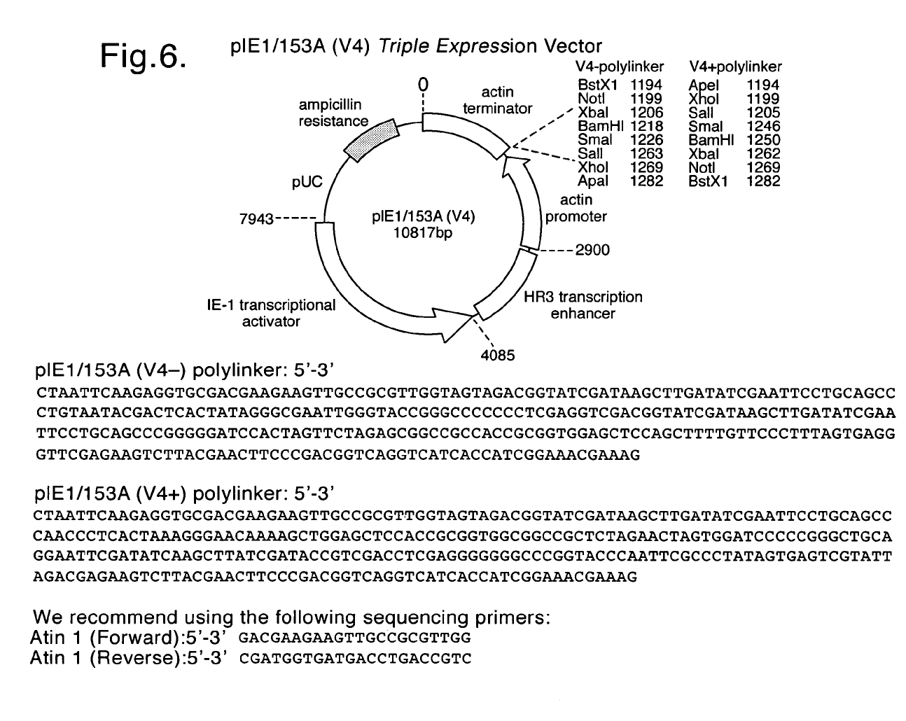

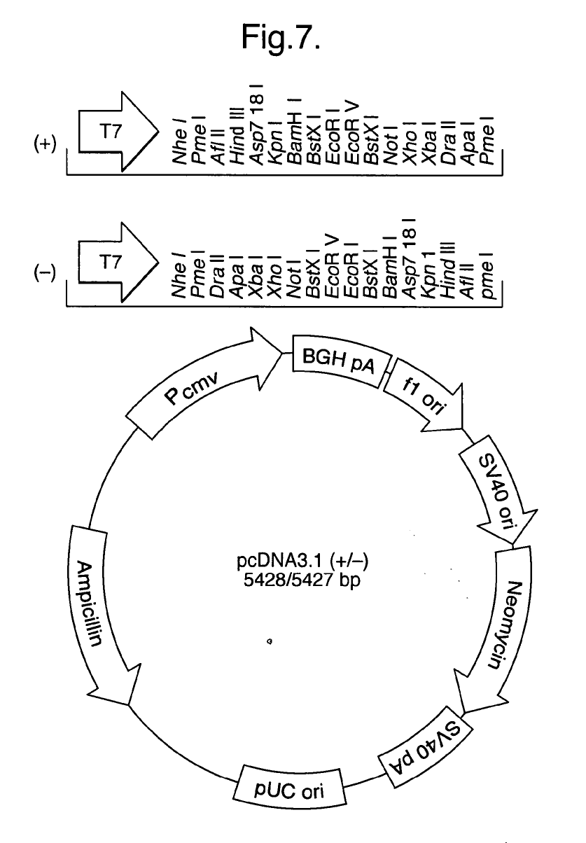

Preferred vectors are piEI/153A (available from Cytostore) and pcDNA3.1 (available from Invitrogen. The maps of these vectors are given in FIGS. 6 and 7.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E. coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end.

Synthetic linkers containing a variety of restriction endonuclease sites are commercially available from a number of sources including International Biotechnologies Inc. New Haven, Conn., USA.

A desirable way to modify the DNA encoding the polypeptide of the invention is to use the polymerase chain reaction as disclosed by Saiki et al (1988) Science 239, 487-491. In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

Exemplary genera of yeast contemplated to be useful in the practice of the present invention are Pichia (Hansenula), Saccharomyces, Kluyveromyces, Candida, Torulopsis, Torulaspora, Schizosaccharomyces, Citeromyces, Pachysolen, Debaromyces, Metschunikowia, Rhodosporidium, Leucosporidium, Botryoascus, Sporidiobolus, Endomycopsis, and the like. Preferred genera are those selected from the group consisting of Pichia (Hansenula), Saccharomyces, Kluyveromyces, Yarrowia and Hansenula. Examples of Saccharomyces spp. are S. cerevisiae, S. italicus and S. rouxii. Examples of Kluyveromyces spp. are K. fragilis and K. lactis. Examples of Pichia (Hansenula) are P. angusta (formerly H. polymorpha), P. anomala, P. pastoris and P. capsulata. Y. lipolytica is an example of a suitable Yarrowia species.

Methods for the transformation of S. cerevisiae are taught generally in EP 251 744, EP 258 067 and WO 90/01063, all of which are incorporated herein by reference.

Suitable promoters for S. cerevisiae include those associated with the PGK1 gene, GAL1 or GAL10 genes, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, α-mating factor pheromone, a-mating factor pheromone, the PRB1 promoter, the GUT2 promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose-repressible fbp1 gene promoter as described by Hoffman & Winston (1990) Genetics 124, 807-816.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADH1 gene is preferred.

In a fourth aspect of the invention there is provided a host cell containing a nucleic acid molecule and/or an expression vector of the second and third aspects of the invention.

Preferably the host cell further displays at its surface, the polypeptide encoded by the nucleic acid molecule and/or an expression vector of the second and third aspects of the invention.

Host cells that have been transformed by the recombinant DNA of the invention are then cultured for a sufficient time and under appropriate conditions known to those skilled in the art in view of the teachings disclosed herein to permit the expression of the polypeptide, which can then be recovered either as soluble polypeptides or as part of a membrane.

The present invention also relates to a host cell transformed with a polynucleotide vector construct of the present invention. The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343).

Preferred eukaryotic host cells include yeast and mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA.

Possible mammalian host cells include Hamster Embryonic Kidney cells, Chinese hamster ovary (CHO) cells available from the ATCC as CCL61, NIH Swiss mouse embryo cells NIH/3T3 available from the ATCC as CRL 1658, and monkey kidney-derived COS-1 cells available from the ATCC as CRL 1650. Possible insect cells are high five and Sf9 cells which can be transfected with baculovirus expression vectors.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (2001) Molecular Cloning, A Laboratory Manual, 3^{rd} Ed. Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming cells and is well known in the art for transforming yeast cell, bacterial cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5×PEB using 6250V per cm at 25 μFD.

Methods for transformation of yeast by electroporation are disclosed in Becker & Guarente (1990) Methods Enzymol. 194, 182.

Physical methods may be used for introducing DNA into animal and plant cells. For example, microinjection uses a very fine pipette to inject DNA molecules directly into the nucleus of the cells to be transformed. Another example involves bombardment of the cells with high-velocity microprojectiles, usually particles of gold or tungsten that have been coated with DNA.

Successfully transformed cells, i.e. cells that contain a DNA construct of the present invention, can be identified by well known techniques. For example, one selection technique involves incorporating into the expression vector a DNA sequence (marker) that codes for a selectable trait in the transformed cell. These markers include dihydrofolate reductase, G418 or neomycin resistance for eukaryotic cell culture, and tetracyclin, kanamycin or ampicillin resistance genes for culturing in E. coli and other bacteria. Alternatively, the gene for such selectable trait can be on another vector, which is used to co-transform the desired host cell.

The marker gene can be used to identify transformants but it is desirable to determine which of the cells contain recombinant DNA molecules and which contain self-ligated vector molecules. This can be achieved by using a cloning vector where insertion of a DNA fragment destroys the integrity of one of the genes present on the molecule. Recombinants can therefore be identified because of loss of function of that gene.

Another method of identifying successfully transformed cells involves growing the cells resulting from the introduction of an expression construct of the present invention to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208. Alternatively, successful transformation can be confirmed by well known immunological methods when the recombinant DNA is capable of directing the expression of the protein. For example, cells successfully transformed with an expression vector produce proteins displaying appropriate calcium exchange function.

In a fifth aspect of the invention there is provided a polypeptide comprising a polypeptide encoded by the nucleic acid molecule of the second aspect of the invention.

As discussed above, the peptide/polypeptide can be expressed from the encoding nucleic acid molecule using expression vectors in host cells. An alternative method of producing peptides is chemical synthesis.

Peptides may be synthesised by the Fmoc-polyamide mode of solid-phase peptide synthesis as disclosed by Lu et al (1981) J. Org. Chem. 46, 3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities.

The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative.

All amino acid derivatives are added as their preformed symmetrical anhydride derivatives with the exception of asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures.

Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesised. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers.

Reagents for peptide synthesis are generally available from Merck/Calbiochem-Novabiochem (UK) Ltd, Nottingham, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

In a sixth aspect of the invention, there is provided a kit of parts comprising:
(i) at least one membrane including at least one polypeptide as defined in the fifth aspect of the invention and/or at least one cell displaying at its surface at least one polypeptide as defined in the fifth aspect of the invention;
(ii) either a solid support to which the at least one membrane and/or the at least one cell may be fixed, or a solution which the at least one membrane and/or the at least one cell may be suspended;
(iii) a multi-welled plate;
(iv) a calcium sensitive detection system (e.g. dye) and
(v) instructions on using the kit

In a seventh aspect of the invention there is provided a use of the compounds isolated in step (i) of the first aspect of the invention in the manufacture of a medicament for the treatment of disease characterised by excessive pigmentation and/or reduced pigmentation and/or in the prevention of sun-induced skin damage.

Preferably the compounds that increase calcium movement can be used in the manufacture of a medicament for treatment of disease characterised by reduced pigmentation and/or for the prevention of sun-induced skin damage.

Alternatively the compounds that reduce calcium movement can be used in the manufacture of a medicament for treatment of disease characterised by elevated pigmentation.

In an eighth aspect of the invention there is provided a use of the compounds isolated in step (i) of the first aspect of the invention in the manufacture of a cosmetic product for increasing and/or reducing skin pigmentation.

Preferably the compounds that increase calcium movement can be used in the manufacture of a cosmetic product for increasing skin pigmentation.

Alternatively the compounds that reduce calcium movement can be used in the manufacture of a cosmetic product for reducing skin pigmentation.

Uses of the compounds identified and isolated using the methods of the invention may be as inhibitors or activators of NCKX5 function.

Inhibitors of NCKX5 can be used as pigmentation inhibitors, for example in darker skin, inhibition of the exchanger will reduce pigment production and lighten the skin, which is desirable in certain Asian societies. Such inhibitors may also enhance UV dependent vitamin D synthesis in skin as a reduction in melanin will reduce melanin-induced blockage of the UV dependent synthesis of vitamin D in skin which has general health benefits including on bone e.g. in improving and/or preventing osteoporosis benefits over time.

Conversely, ingredients that activate the exchanger can be used to enhance pigment production in skin. For example, consumers with lighter skin may obtain a natural tan with out the risks that arise from sun exposure (such as burning and the discomfort associated with that, skin ageing and skin cancer). The activators might also use a tanning product to prepare the skin for subsequent sun exposure, a so-called pre-sun treatment. Use of such a tanning product may reduce the effects over time of photoageing, and therefore indirectly will have a skin ageing benefit. For example wrinkles, sallowness, sagging, fine lines, age spots, mottled pigmentation could be reduced. Individuals who are especially sensitive to UV and/or visible light, may use a tanning product to better protect themselves from sun exposure when they go outside; for example patients with porphyria or xeroderma pigmentosum.

The protection of such activators may extend to individuals for which sun exposure is contra-indicated e.g. those who are especially sensitive to skin cancer, for example because of defective DNA repair mechanisms or phototoxic reactions.

Inhibitors and activators of SLC24A5 might also be used to change the pigmentation or composition of skin and/or hair melanin so that the skin and/or hair colour is altered.

Inhibitors and activators of SLC24A5 might also be used to change the pigmentation in animals so that the coat or skin colour of the animal is lightened or darkened. The uses of pigmentation changes in animals may range from protection of animals against sunburn to altering skin pigmentation for textiles such as leather.

In a ninth aspect of the invention there is provided a compound identified by the method of the first aspect of the invention.

### Meanings of Terms Used

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). It is contemplated that where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “polypeptide” or “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. To be active, any polypeptide must have sufficient length to display biological and/or immunological activity.

The term “functionally equivalent variants” as used herein refers to a protein wherein at one or more positions there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr.

Functionally equivalent variants can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 85% sequence identity, more preferably at least 90% sequence identity, more preferably at least 95% sequence identity, more preferably at least 98% sequence identity, and most preferably at least 99% sequence identity. Substantially equivalent nucleic acid molecule of the invention can have lower percent sequence identities, taking into account, for example, the redundancy or degeneracy of the genetic code. Preferably, the nucleic acid molecule has at least about 65% identity, more preferably at least about 75% identity, more preferably at least about 80% sequence identity, more preferably at least 85% sequence identity, more preferably at least 90% sequence identity, more preferably at least about 95% sequence identity, more preferably at least 98% sequence identity, and most preferably at least 99% sequence identity. For the purposes of the present invention, sequences having substantially equivalent biological activity and substantially equivalent expression characteristics are considered substantially equivalent.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res 22, 4673-80). The parameters used may be as follows:

Fast pairwise alignment parameters: K-tuple (word) size; 1, window size; 5, gap penalty; 3, number of top diagonals; 5.
Scoring method: ×percent.
Multiple alignment parameters: gap open penalty; 10, gap extension penalty; 0.05.
Scoring matrix: BLOSUM.

The term “functionally equivalent fusions” as used herein denotes a polypeptide of the invention operatively linked to another polypeptide. Within a fusion protein the polypeptide according to the invention can correspond to all or a portion of a protein according to the invention. In one embodiment, a fusion protein comprises at least one biologically active portion of a protein according to the invention. In another embodiment, a fusion protein comprises at least two biologically active portions of a protein according to the invention. Within the fusion protein, the term “operatively linked” is intended to indicate that the polypeptide according to the invention and the other polypeptide are fused in-frame to each other. The polypeptide can be fused to the N-terminus or C-terminus, or to the middle. The basic properties of the polypeptide of the invention have not significantly been changed. “Significantly” in this context means that one skilled in the art would say that the properties of the variant may still be different but would not be unobvious over the ones of the original protein.

The term “functionally equivalent derivatives” as used herein denotes a fragment or modified version of a parent polypeptide. The derivative may be modified by the addition of one or more naturally or non-naturally occurring amino acids or other molecules e.g. to facilitate coupling the polypeptide to another peptide or polypeptide, to a large carrier protein or to a solid support or its insertion into a membrane (e.g. the amino acids tyrosine, lysine, glutamic acid, aspartic acid, cysteine and derivatives thereof, NH2-acetyl groups or COOH-terminal amido groups, amongst others) and the basic properties of at least one of the parent polypeptides have not significantly been changed. “Significantly” in this context means that one skilled in the art would say that the properties of the variant may still be different but would not be unobvious over the ones of the original protein.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “expression vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters and often enhancers, (2) a structural or coding sequence which is transcribed into mRNA and translated into protein, and (3) appropriate transcription and translation initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “compartment” refers to a discrete 3D area that may or may not contain a solution and/or solid matter. In the context of this invention a compartment may include the inside of a cell, the solution surrounding the cell, a contained space on one side of a membrane e.g. a well on a multiwelled plate.

The term “membrane” denotes any thin, typically planar structure or material that separates two environments. Membranes of the invention include biological membranes (lipid bilayer membranes) such as cell membranes, membranes of cell organelles and artificial membranes capable of supporting an inserted protein e.g. polymeric membranes.

The term “altering the composition” denotes altering the colour and or melanin composition of hair and. or skin by using inhibitors and activators of NCKX. The melanin composition of the hair and/or skin is determined by the proportions of eumelanin and pheomelanin that is present in the tissue.

### PREFERRED EMBODIMENTS

Examples embodying certain preferred aspects of the invention will now be described with reference to the following figures in which: —

FIG. 1—NCKX5 (SLC24A5) transcript reference sequence. Nucleotide and amino acid sequence of NCKX5 showing the position of the Ala/Thr SNP linked to skin pigmentation.

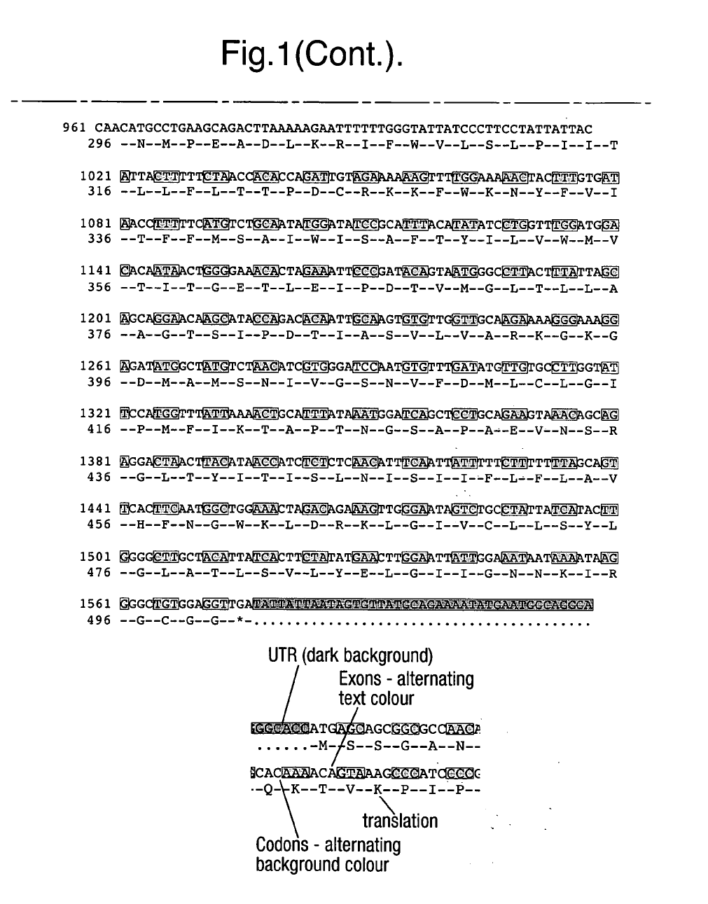

FIG. 2—Distribution of alleles in relation to human skin pigmentation. Graph showing the frequency of alleles and relationship skin colour The reference allele is the Threonine containing and the alternate copy is Alanine containing. Hence, Alanine allele is shown to be predominantly found in darker skins.

FIG. 3—NCKX5 homology and position of Ala/Thr variation

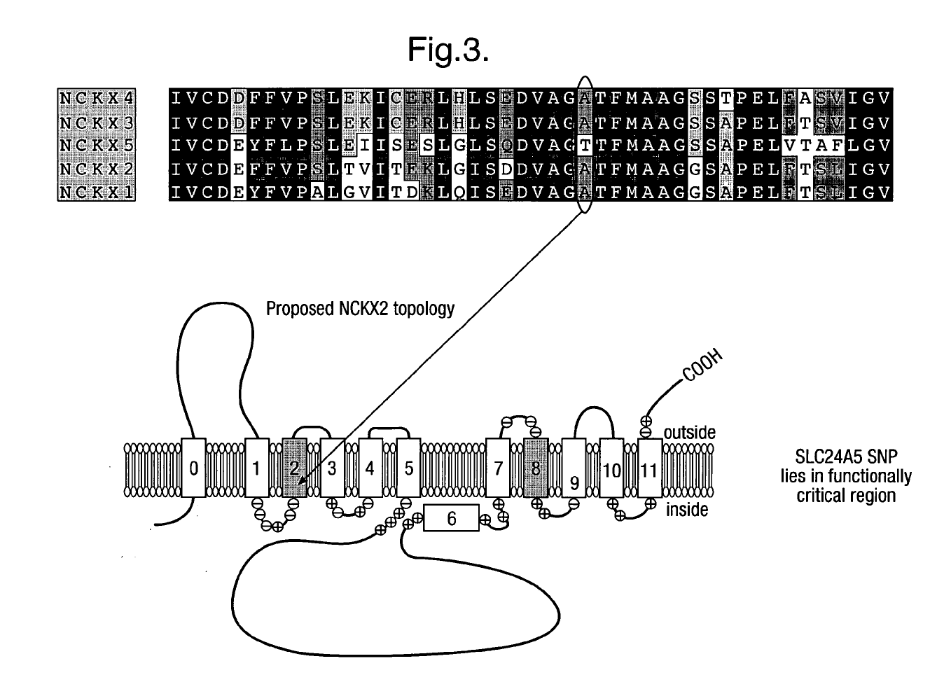

FIG. 4—Calcium extrusion using modified NCKX2. The figure shows the amount of calcium extrusion due to the two variants (Ala and Thr) of an NCKX2 molecule that has been modified to resemble NCKX5. Ala177 (equivalent to Ala111 in NCKX5) in NCKX2 (dark skin allele) is associated with increased calcium exchange.

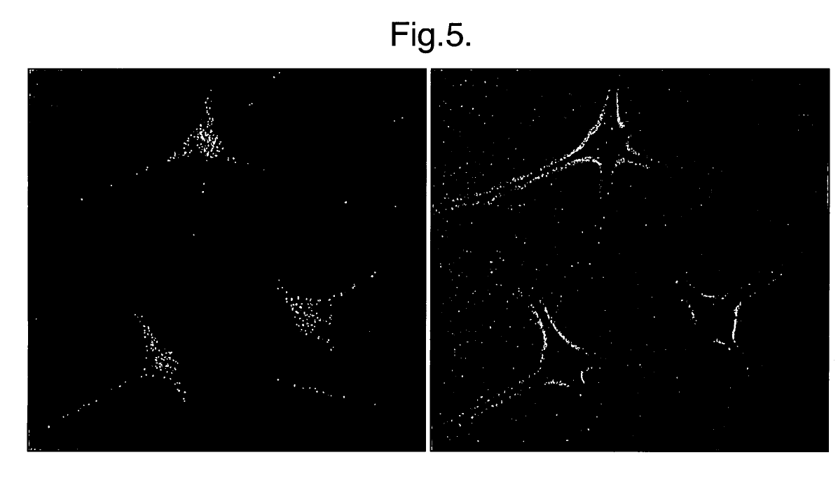

FIG. 5—Staining of melanocytes. Melanocytes stained with NCKX5 antisera show punctuate cytoplasmic staining and peri-nuclear staining.

FIG. 6—pIE1/1534 vector map

FIG. 7—pcDNA3.1 vector map

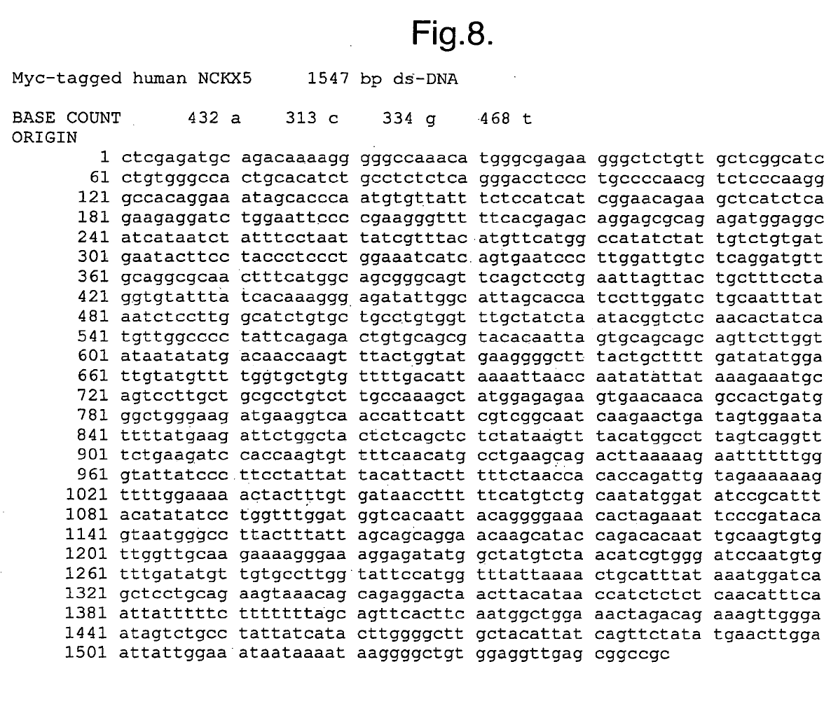

FIG. 8—Myc tagged human NCKX5 nucleotide sequence

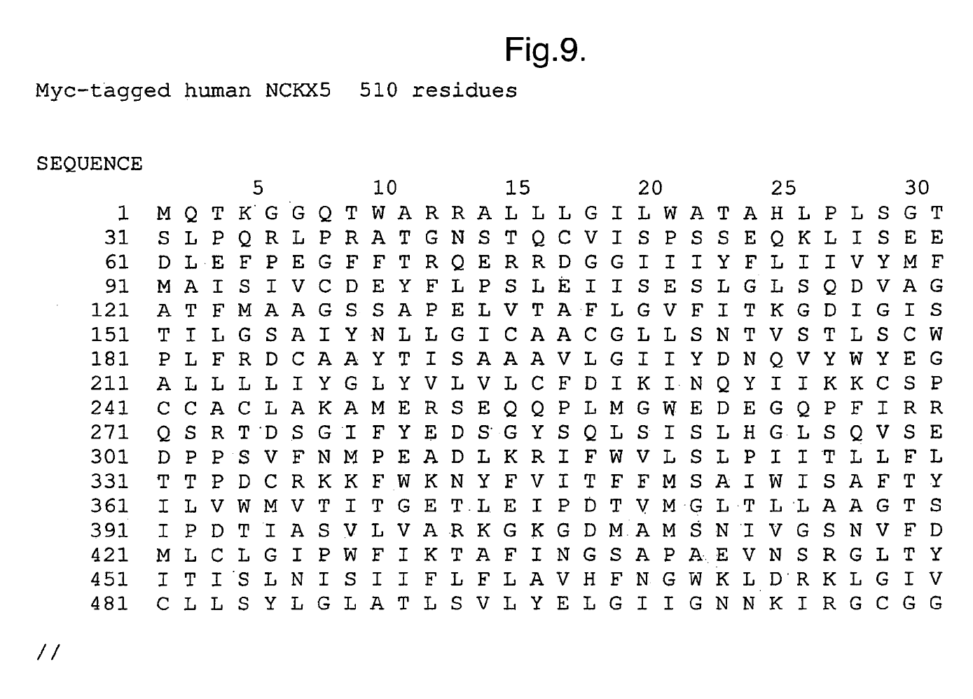

FIG. 9—Myc tagged human NCKX5 amino acid sequence

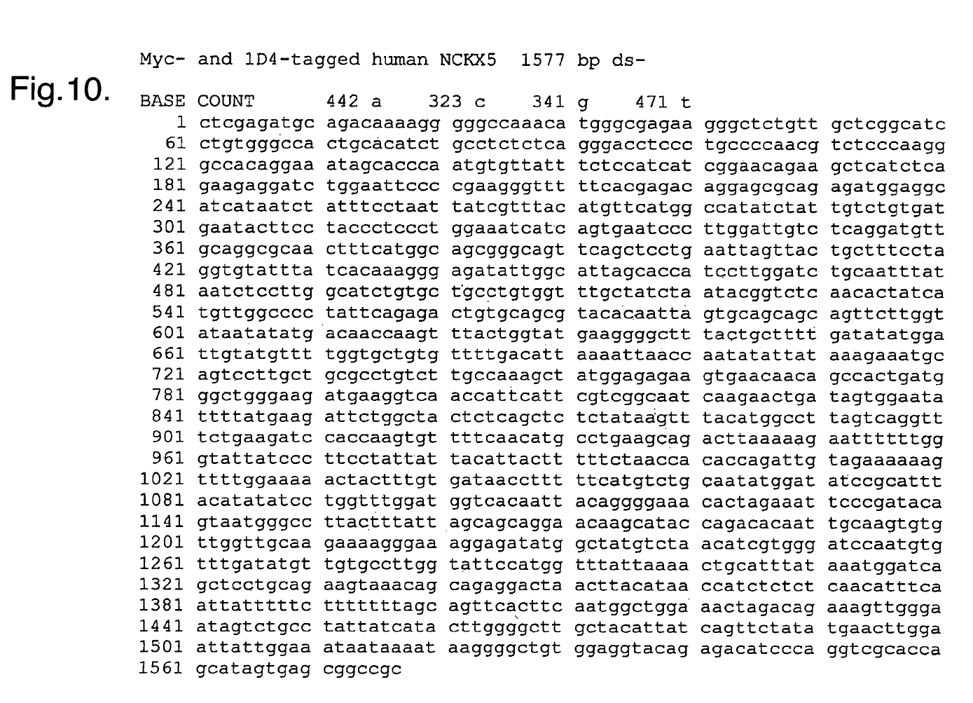

FIG. 10—Myc and 1D4 tagged human NCKX5 nucleotide sequence

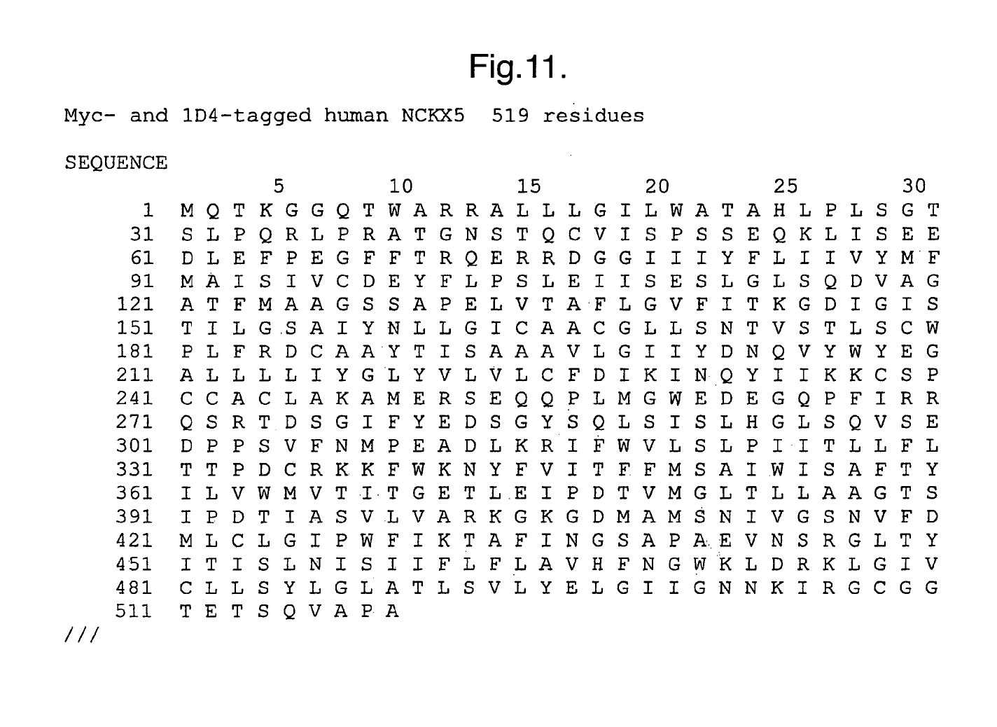

FIG. 11—Myc and 1D4 tagged human NCKX5 amino acid sequence

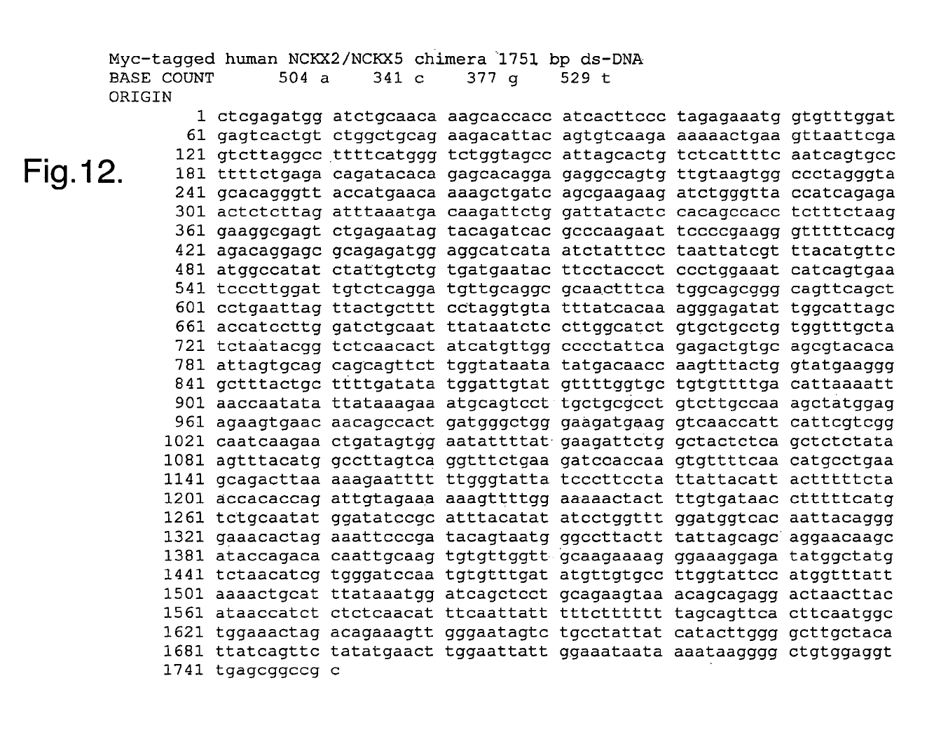

FIG. 12—Myc tagged human NCKX2/NCKX5 chimera nucleotide sequence

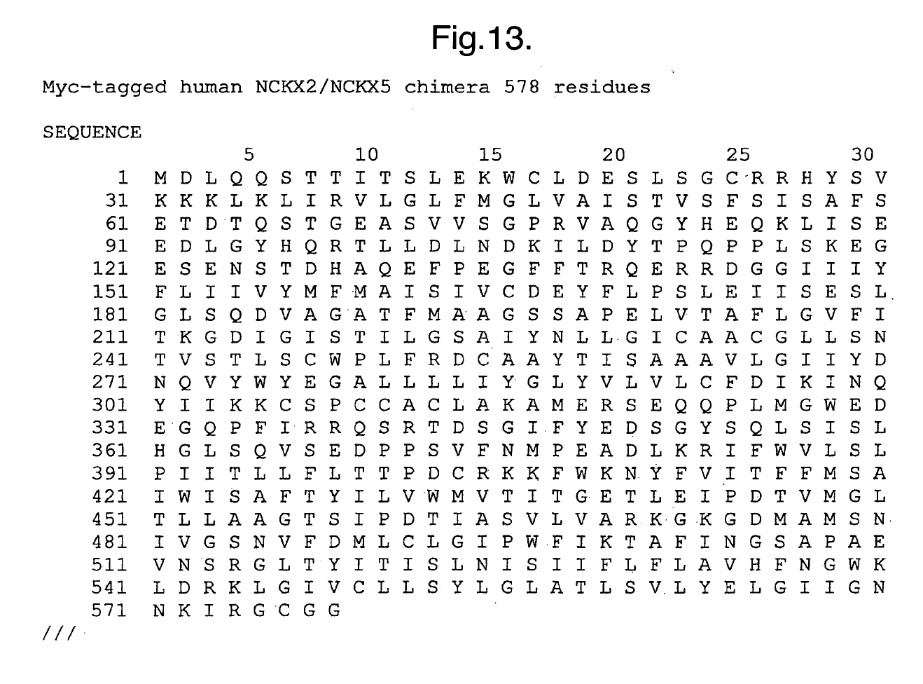

FIG. 13—Myc tagged human NCKX2/NCKX5 chimera amino acid sequence

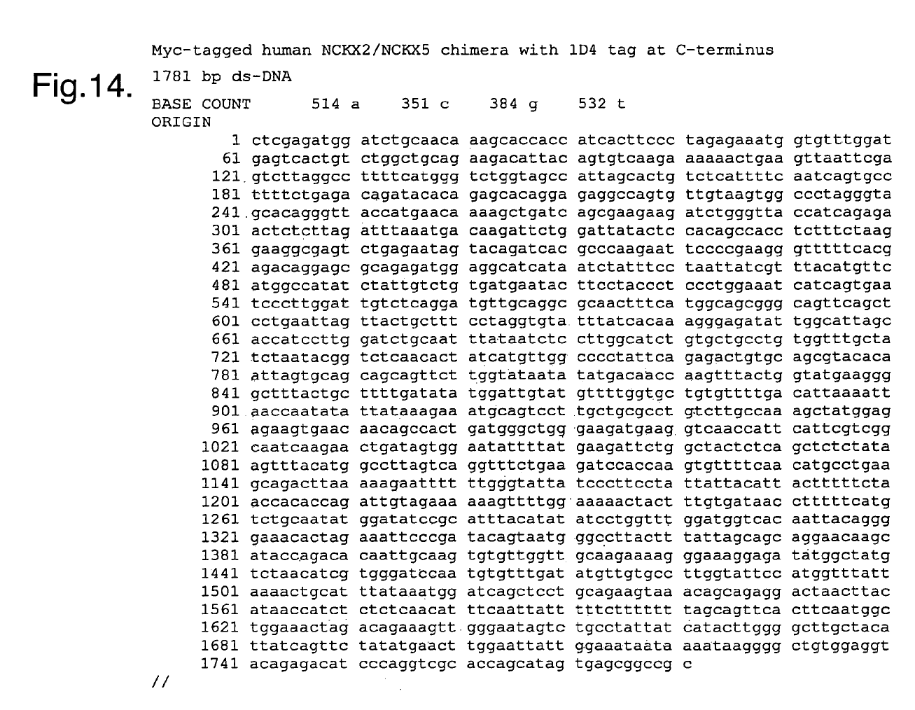

FIG. 14—Myc tagged human NCKX2/NCKX5 chimera nucleic acid molecule with ID4 tag at C-terminus

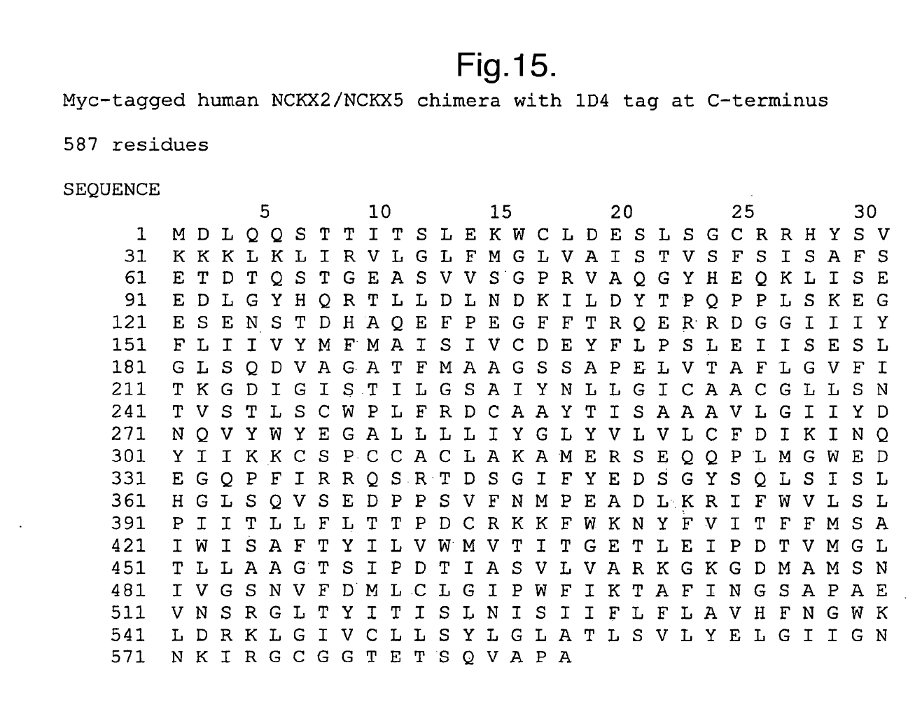

FIG. 15—Myc tagged human NCKX2/NCKX5 chimera amino acid sequence with ID4 tag at C-terminus

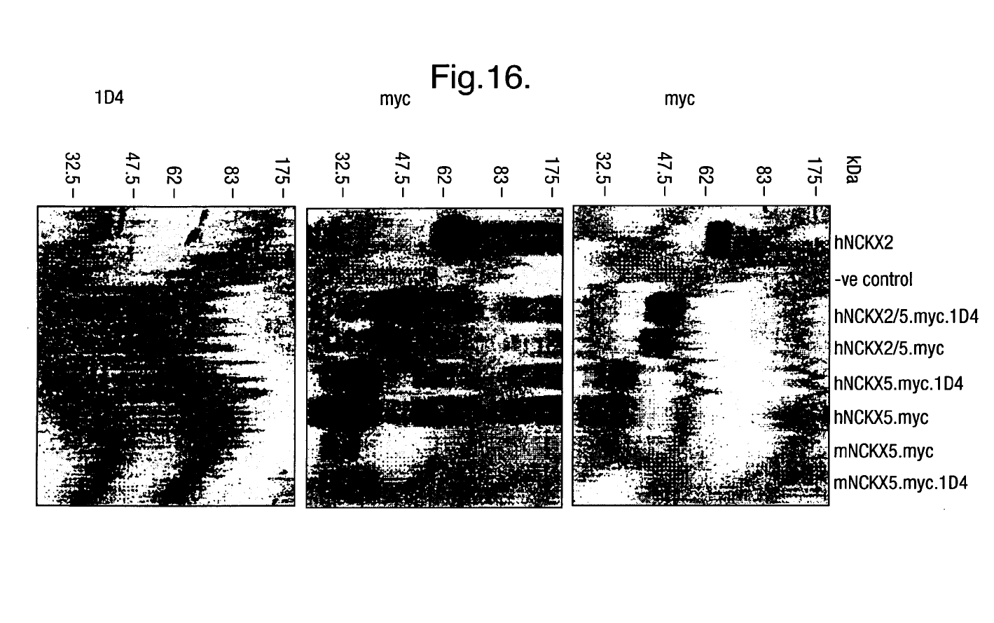

FIG. 16—Western blot of NCKX5 expression (Includes NCKX5 and NCKX5/NCKX2 chimeras and 1D4 and myc tagged versions)

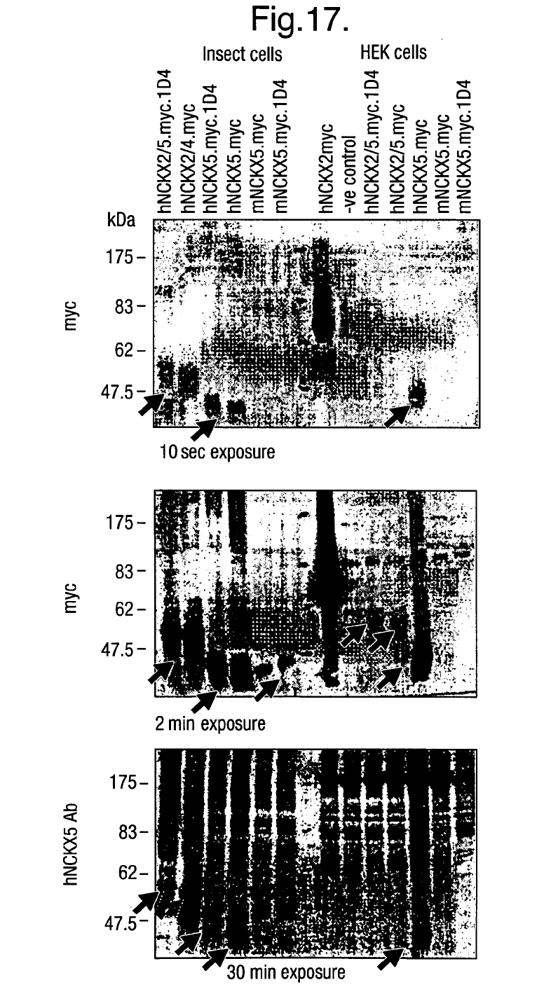

FIG. 17—Western blot of NCKX5 expression (Includes NCKX5 and NCKX5/NCKX2 chimeras and 1D4 and myc tagged versions) in High five insect cells and HEK cells.

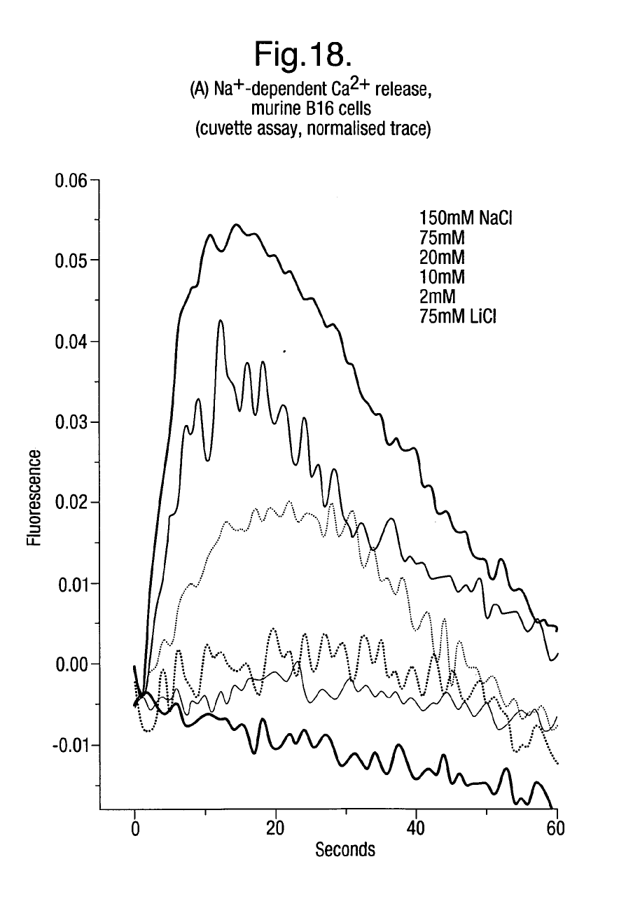

FIG. 18—Na+ dose-dependently induces intracellular Ca2+ release in B16 cells. Detected by cuvette method.

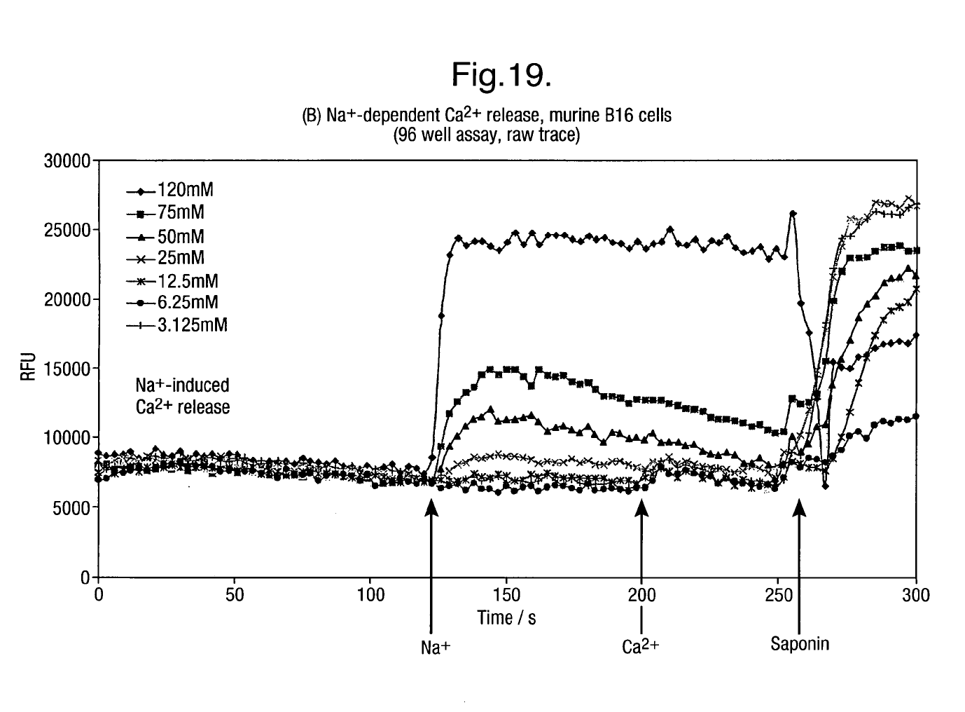

FIG. 19—Na+ dose-dependently induces intracellular Ca2+ release in B16 cells.—Detected by 96 well assay

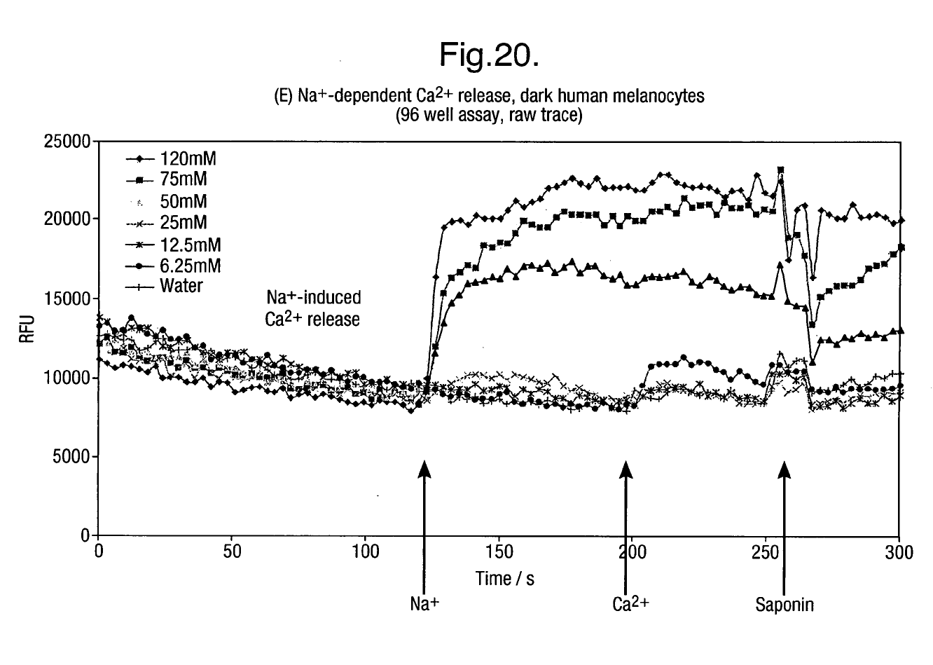

FIG. 20—Na+ dose-dependently induces intracellular Ca2+ release in B16 cells. Dose-dependent Na+-induced release of intracellular Ca2+ in dark human melanocytes by 96 well method.

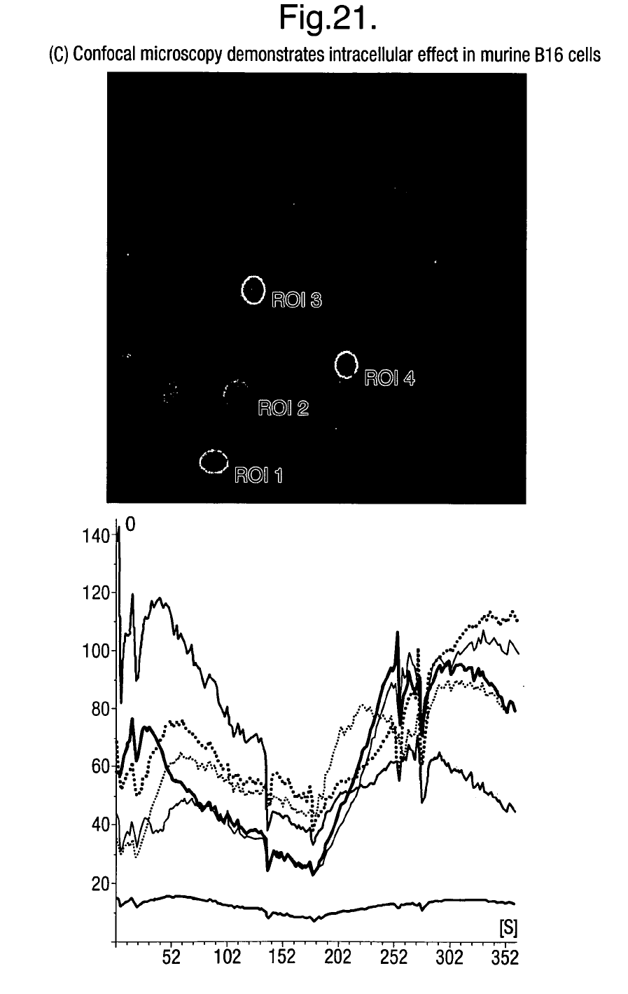

FIG. 21—Na+ dose-dependently induces intracellular Ca2+ release in B16 cells. Detected by confocal microscopy.

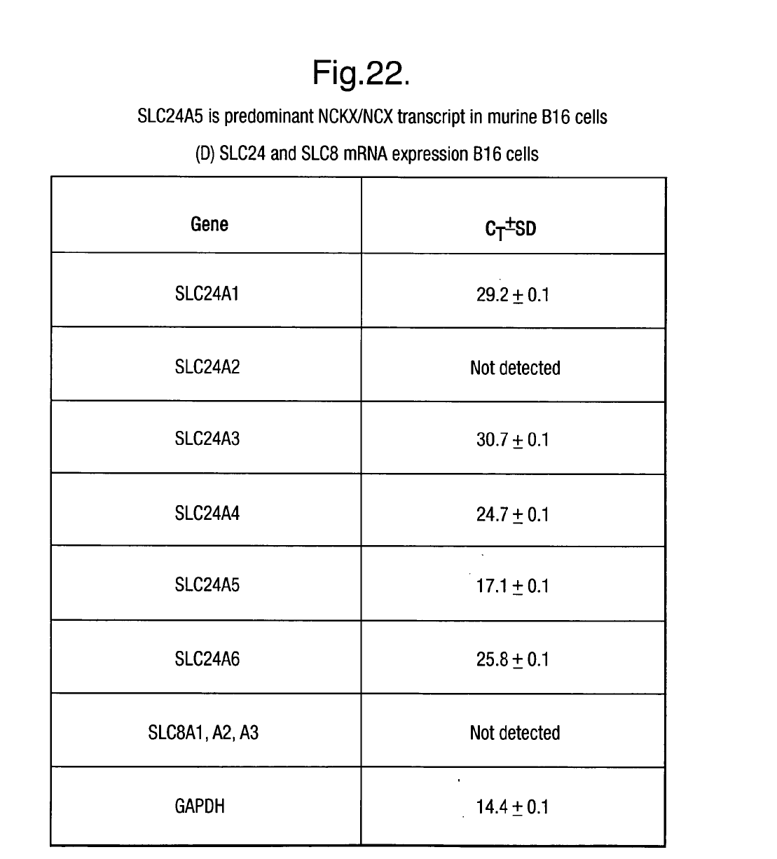

FIG. 22—Na+ dose-dependently induces intracellular Ca2+ release in B16 cells. mRNA expression of SLC24 and SLC8 in B16 cells as detected by real-time PCR following reverse-transcription of 1 μg RNA.

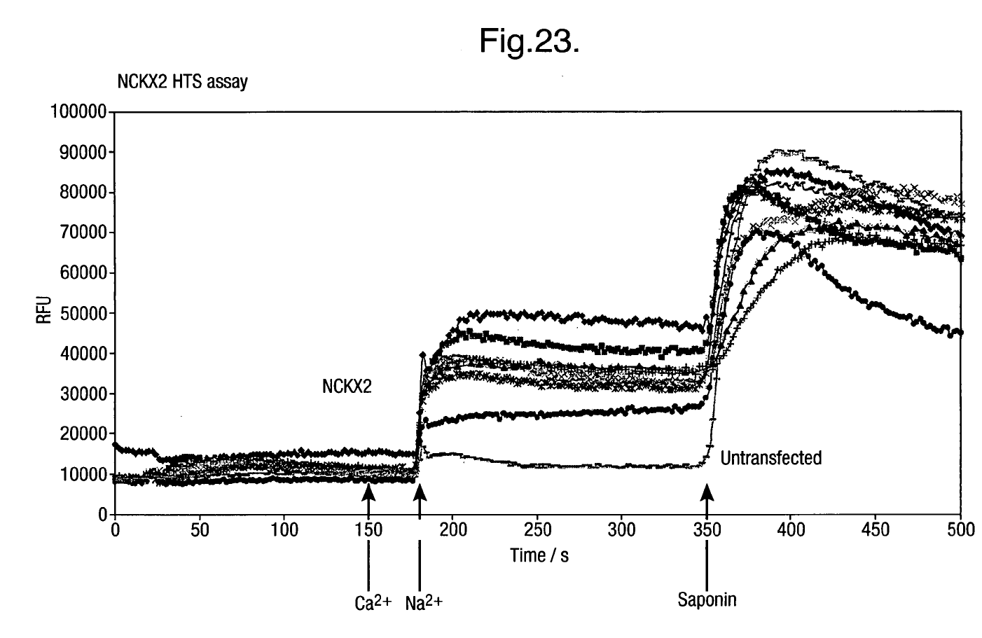

FIG. 23—A heterologous NCKX2 assay.

A heterologous NCKX2 assay scaled to high throughput screening format being tested in the Amaxa 96 well Nucleofection device.

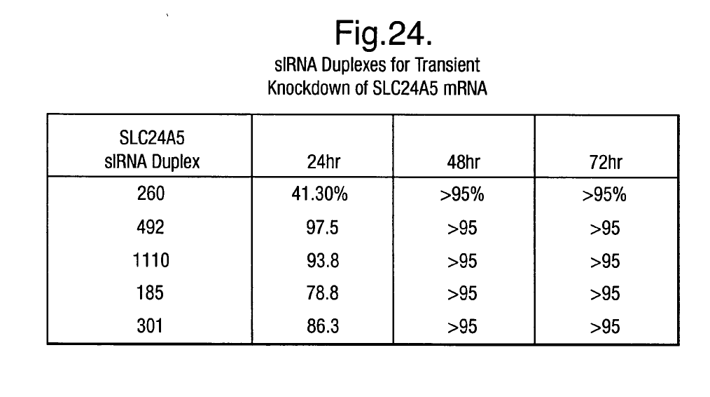

FIG. 24—Table showing decrease in SLC24A5 levels.

5 separate siRNA duplexes (designed by Invitrogen) decrease SLC24A5 mRNA levels by 95% or more in human primary melanocytes. This effect can be maintained for up to 10 days with re-transfection (data not shown).

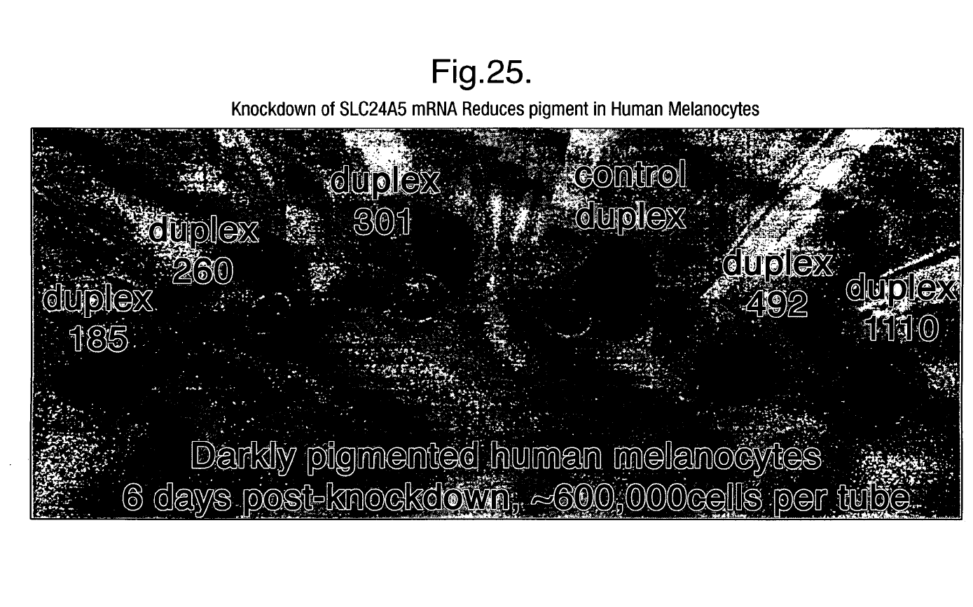

FIG. 25—Knockdown of SLC24A5 mRNA reduces pigment in human melanocytes

SLC24A5 knockdown followed by normalisation of cell numbers (by coulter counting) and centrifugation to pellet the melanocytes provides qualitative evidence that SLC24A5 is involved in a melanogenic process.

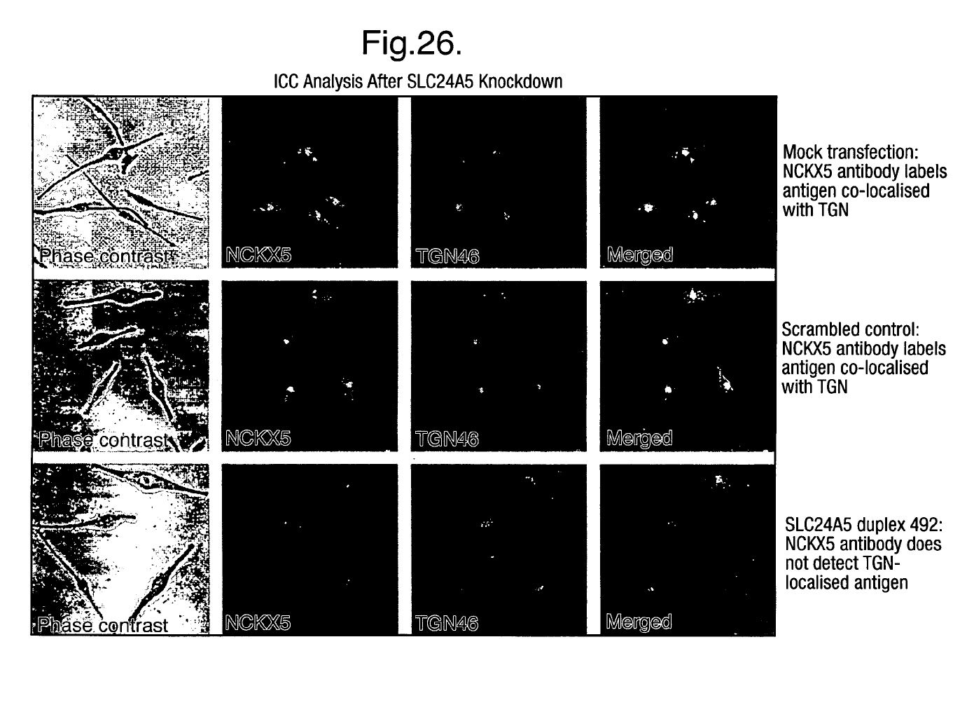

FIG. 26—ICC Analysis after SLC24A5 knockdown

A 5 day knock down (using duplex 492 and controls) was conducted using darkly pigmented primary human melanocytes (Cascade). Primary antibodies for detection: Rabbit anti-NCKX5 cytosolic loop (raised using the peptide sequence DEGQPFIRRQSRTDSG) and sheep pAb anti-TGN46; Labelling was via Alexa Fluor® 488 anti-rabbit and 633 anti-sheep IgG's. The anti-NCKX5 polyclonal antibody localises within the TGN.

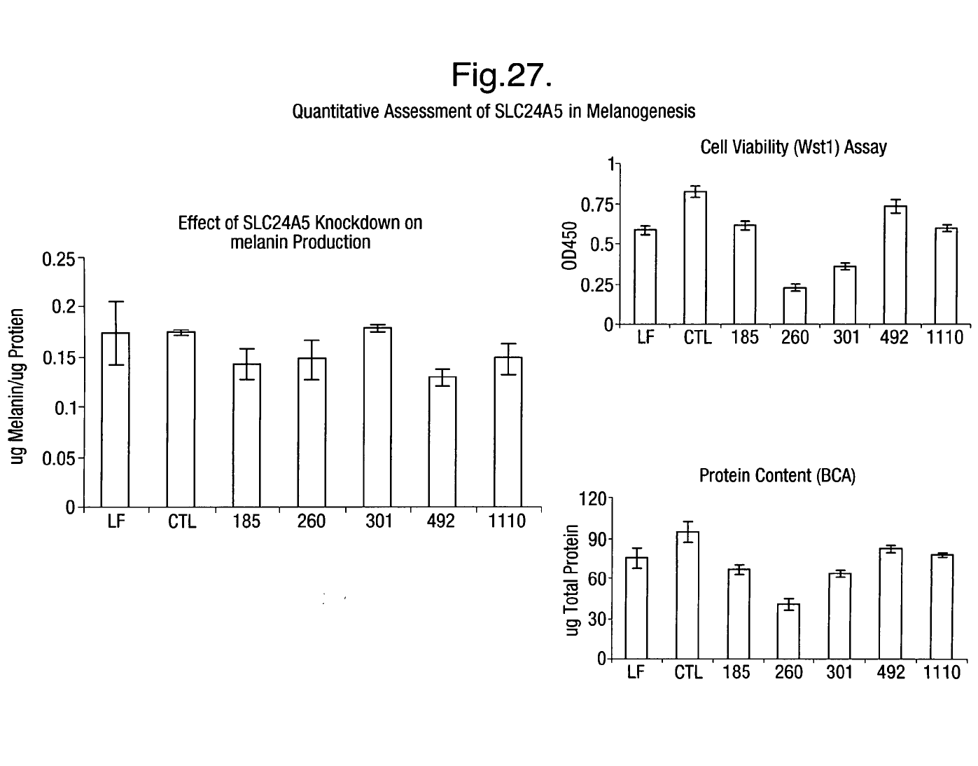

FIG. 27—Quantitative assessment of melanogenesis

A decrease in post-treatment melanin content is noted for duplex (492) in lightly pigmented human melanocytes (replicated ×3).

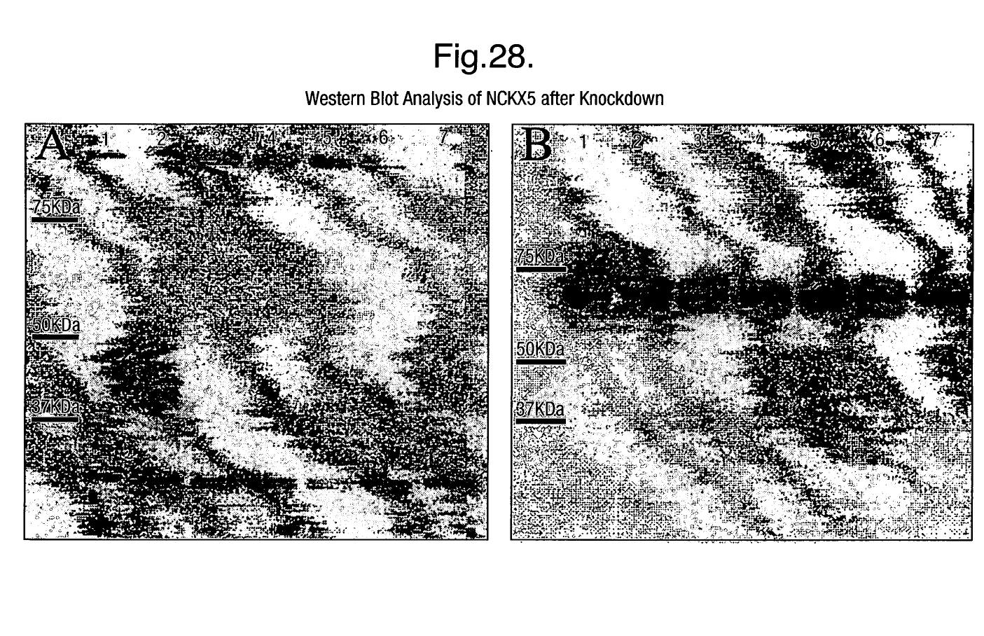

FIG. 28—Western blot analysis of NCK5 after knockdown

Using western blot we have investigated NCKX5 protein expression 5 days after siRNA mediated knockdown (all 5 siRNA duplexes and controls were tested). Western blot was also used to evaluate the expression and processing (maturation) of tyrosinase (tyr) in the same samples. SDS-PAGE (10% PA for NCKX5, 4-12% for tyr, 5 μg protein/lane). A) Western blot for NCKX5: Detection via rabbit anti-NCKX5 pAb (anti C-terminus peptide). B) Western blot for tyr, via goat pAb (Santa Cruz). 1=No treatment, 2=scrambled siRNA control, 3-7=siRNA's 185, 260, 301, 492, 1110. Western blot A) shows 2 bands, of approx. 42 and 44 KDa (significantly smaller than the predicted m.w. for NCKX5) in control wells. These bands are absent in the samples where SLC24A5-specific duplexes were used (lanes 3-7). It is possible that the doublet represents the presence of alternate SLC24A5 splice variants. The expression and processing of tyrosinase after knockdown (B) suggests that NCKX5 does not grossly alter the expression and/or maturation of tyrosinase, the rate limiting enzyme for melanin biosynthesis.

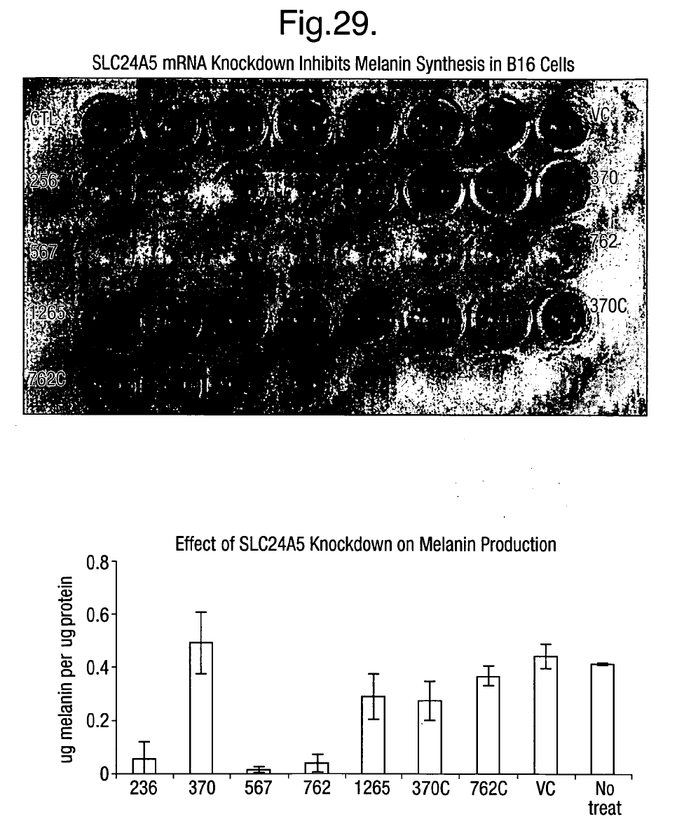

FIG. 29—SLC24A5 mRNA knockdown inhibits melanin synthesis in B16 cells

Cells were transfected (lipofectamine 2000) with siRNA duplexes or controls for 8 hours. Cells were cultured for a further 3 days prior to analyses.

Media was removed and melanin quantified by OD450. Cell viability measured using Wst1 proliferation reagent (Roche) before lysis using 1% triton ×100. Protein content of each well was determined by BCA assay.

Experiment (each in quadruplicate) reproduced x3 consecutively. 3 siRNA duplexes (256, 567 and 762) visibly decreased melanin synthesis.

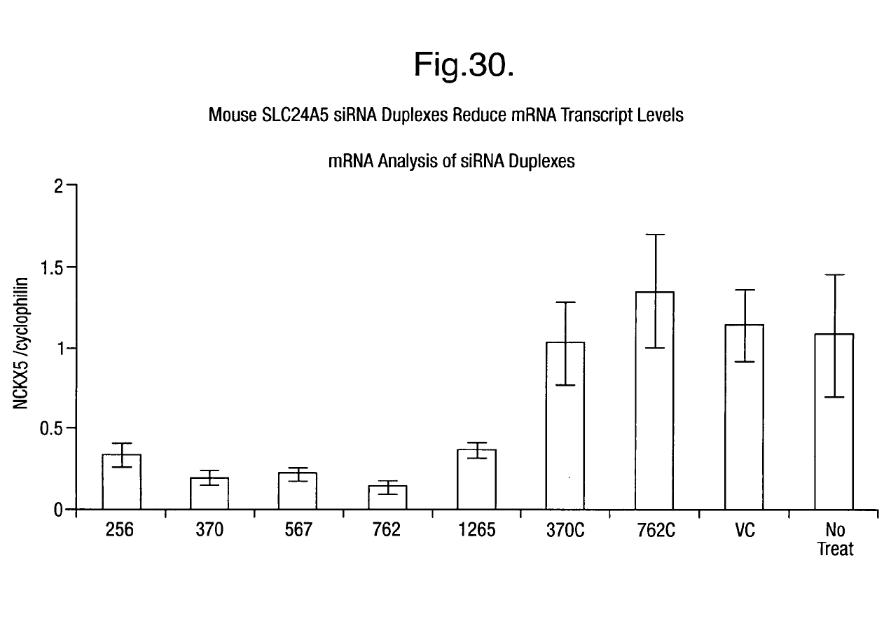

FIG. 30—Mouse SLC24A5 siRNA Duplexes Reduce mRNA Transcript Levels

All 5 siRNA duplexes were shown to be capable of achieving SLC24A5 mRNA knockdown in mouse B16 melanocytes. 3 duplexes were shown to be capable of reducing mRNA levels by more than 80% under the test conditions. Cells were transfected (Lipofectamine 2000) with 50 nM siRNA duplex or control for 8 hours. Cells were cultured for a further 24 hours prior to harvesting and real-time PCR analyses.

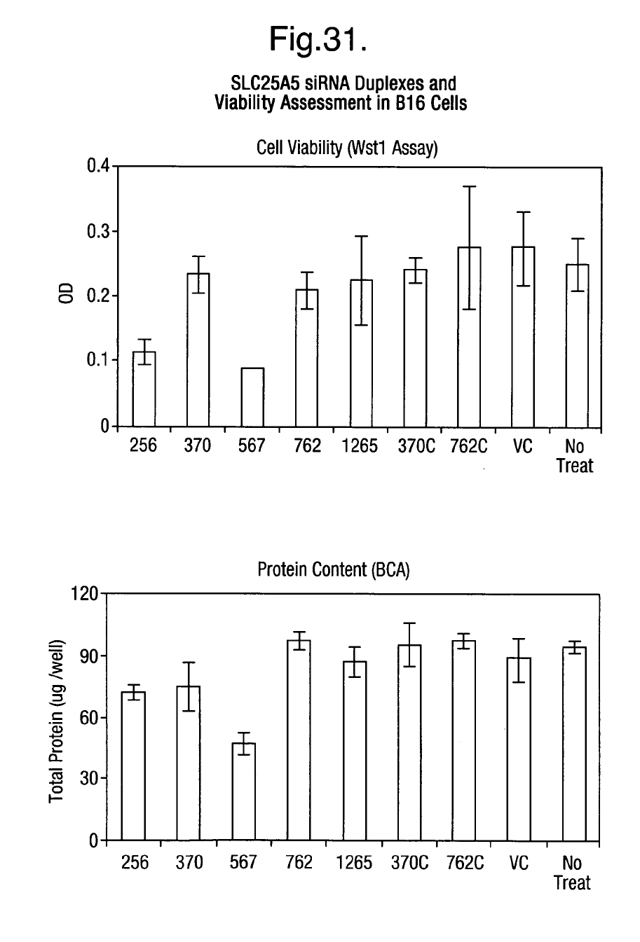

FIG. 31—SLC24A5 siRNA Duplexes and Viability Assessment in B16 Cells

Cell viability and protein content determination post-treatment could help to identify the manner in which SLC24A5 modulates pigment production in B16 melanocytes. Cell viability assessment at the end of each experiment suggested significant toxicity associated with the use of 2 duplexes (256 and 567). This conclusion is supported by data obtained for protein content. Duplex 762 appeared to reduce pigment production without affecting viability under the experimental conditions tested.

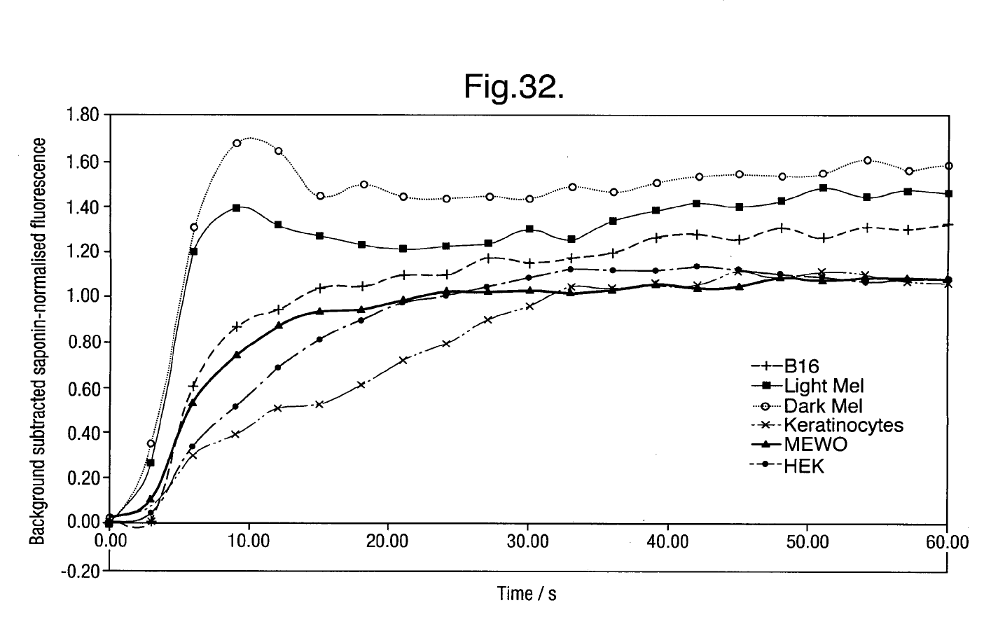

FIG. 32—Sodium-induced intracellular calcium release in various cells

100,000 cells were plated overnight into a 96 well plate in triplicate. Intracellular calcium release in response to sodium was measured as described in methods. Data are background subtracted and saponin-normalised for comparison. Data are mean of 3 replicates.

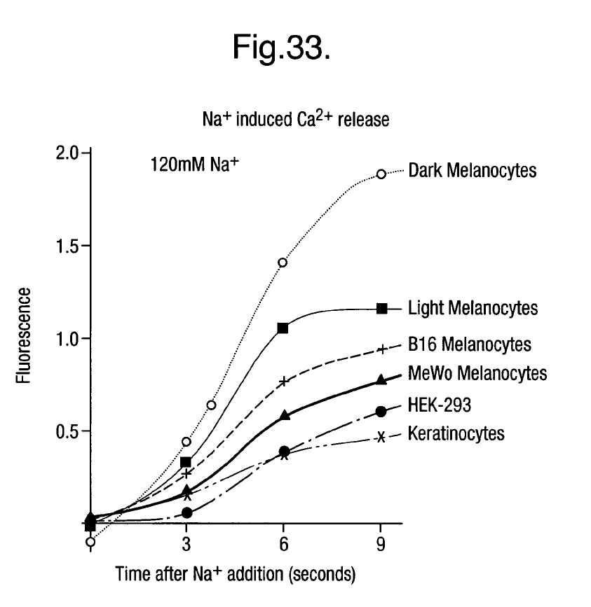

FIG. 33-Sodium-induced intracellular calcium release in various cells

100,000 cells were plated overnight into a 96 well plate in triplicate. Intracellular calcium release in response to sodium was measured as described in methods. Data are background subtracted and saponin-normalised for comparison. Data are mean of 3 replicates.

### EXAMPLE 1

### Distribution of NCKX5 in Individuals of S. Asian Ancestry

Studies have shown that in a sample of individuals of UK volunteers having S Asian ancestry there is a correlation between different allelic versions of NCKX5 and skin colour (described in a co-pending US application from the applicant). NCKX5 has a single nucleotide polymorphism that encodes variation at amino acid position 111 and in dark skins there is a predominance of the Ala111 amino-acid residue, whilst in lighter skins there is a predominance of the Thr111 amino-acid residue. FIG. 2 shows the distribution of these two alleles in darker and lighter skin types, from a total of 230 volunteers of South Asian descent. Volunteers were selected by taking measurements of non-sun exposed skin using a chromameter. The L* reading of the chromameter gives a direct read out of the reflectance of skin, which in turn is directly related to the melanin content of the skin. Volunteers who fell into the 20% ‘extreme’ tails of the skin colour distribution were genotyped. These volunteers therefore had either lighter or darker skin colour compared to the average. The allele frequency difference for the non-synonymous polymorphism at aminoacid 111 of NCKX5 is 39% with an extremely low chance of false discovery (False Discovery Rate=9.7×10⁻¹³).

### EXAMPLE 2

### Activity of NCKX5

The activity of NCKX5 and the effect of the two alleles (Ala111 and Thr111) on Sodium-Potassium/Calcium exchange was investigated by mutating an NCKX exchanger (NCKX2) that is naturally found inserted in retinal rod photoreceptor cell membranes to more closely resemble NCKX5.

The method of producing these constructs is as described in Winkfein et al. (2003) Biochemistry vol 42 pg 543-552 and in investigating their effects as per Kang et al (2005) J Biiol chem. vol 280 pg 6823-6833.

The mutated NCKX2 was also modified to two forms by including the NCKX5 SNP (from position 111), at NCKX2 residue 177 i.e. one NCKX2 form had an Ala177 and the other form a Thr177 (See FIG. 3 for the position of the variant in the three dimensional structure of the protein and a comparison of the sequence identity of the NCKX family)

The modified NCKX2 was expressed and the calcium exchange function of the molecule was measured. It was found that the Thr177 version that is associated with light skin showed a significantly reduced calcium exchange in comparison to the Ala177 version which is associated with dark skin. (See FIG. 4)

### EXAMPLE 3

### NCKX5 Transcript and Localisation Analysis

### Transcript Expression

### Method:

mRNA levels were measured using real-time PCR with Taqman probes (purchased from Applied Biosystems) and normalised to housekeeper gene human transcription factor IID TATA box binding protein (huTBP).

NCKX pre-designed and validated sybr primers from Qiagen: 5196, 5197, 5198, 5199, 5200, 5201, 5202, 5203, 5204, 5205, 5206, 5207. The primers/probes used were:
NCKX5 ABI primer/probe set: hs01385406_g1, spanning exons 3-4 FAM-linked
huTBP ABI primer/probe set 4326322E, VIC-linked

### Materials:

cDNA was derived from cultured melanocytes, fibroblasts and keratinocytes isolated from donors of Indian origin with various skin colours and also commercially sourced from Caucasian and Negroid donors

The cDNA derived from Indian volunteers was derived from skin biopsies. Commercially sourced cDNA was derived from different human body tissues (brain, colon, kidney, lung, muscle, stomach and uterus).

### Findings:

NCKX5 mRNA transcript was detected in all the cultured melanocytes and skin biopsies tested. The NCKX5 mRNA expression levels in skin biopsies do not appear to correlate with colour differences, ethnic origin or SLC24A5 genotype in a small cohort tested (n=22)

NCKX5 mRNA was not detectable by real-time PCR using Taqman probes in the non-skin tissues (brain, colon, kidney, lung, muscle, stomach, and uterus).

SLC24A5 mRNA was not detectable by real-time PCR using Taqman probes in cultured fibroblasts or keratinocytes (i.e. skin cells other than melanocytes), however evidence, using Sybr green detection method (a realtime PCR method using qiagen quantitect kit using a Biorad icycler), that low levels of SLC24A5 mRNA may be present in cultures of dermal fibroblasts (at much lower levels than cultured melanocytes from the same donor.)

In cultured melanocytes NCKX5 mRNA levels are higher than NCKX1 (SLC24A1) mRNA levels. NCKX4 (SLC24A4) and NCKX6 (SLC24A6) mRNA is detectable in cultured melanocytes, but at lower levels than NCKX5 or NCKX1 (SLC24A1), using the Sybr green detection method.

### Protein Localisation

### Tools:

Rabbit polyclonal antisera were raised against 2 peptides fragments derived from NCKX5 (one in the predicted large hydrophilic loop and one in the carboxy-terminus of the protein). Antibodies were affinity purified against the peptides.

The antibodies were generated by Eurogenetec using the following protocol:

Primary immunisation of 2 rabbits with both peptide fragments followed by 3 subsequent boosted immunisations 2, 4 and 8 weeks after primary immunisation. Peptides were conjugated to keyhole limpet haemocyanin and the final bleed was collected 12 weeks after the initial immunisation. For affinity purification, each peptide was conjugated to spharose and 5 ml of final bleed sera was incubated with it in batch, packed into a column, washed in PBS and eluted into 100 mM glycine-HCL pH2.5. The elute was neutralised in 1M Tris at pH9 abd buffer exchanged into PBSA.

Secondary detection was conducted using donkey anti-rabbit Alexa-fluor 488 (Invitrogen).

Visualisation of the bound antibodies was conducted using by confocal fluorescence microscopy.

### Material:

Cultured primary human melanocytes from Caucasian or negroid donors were grown on glass cover-slips and fixed in 2% paraformaldehyde and permeabilised with 0.5% Saponin.

### Findings:

No evidence of plasma membrane staining indicating that NCKX5 was not localised in the plasma membrane.

Punctate staining was found throughout cell. indicating the presence of NCKX5 in the melanocyte.

(See FIG. 5, for staining)

### EXAMPLE 4

### Construction of NCKX5 Expression Vector

All clones were inserted between the XhoI/NotI DNA-restriction sites in either vectors pEIA and pcDNA3.1 The pIEA vector is available from Cytostore as the TriplExpress vector (www.cytostore.com) and its structure and sequence is shown in FIG. 6. The PCDNA3.1 vector may be purchased from Invitrogen (www.invitrogen.com) and its structure is shown in FIG. 7.

DNA encoding the NCKX5 amino acid sequence (amino acids 63-64 EF; (GAGTTT)) was modified using standard recombinant methods to introduce an ECOR1 DNA restriction site (GAATTC).

The insertion of the ECOR1 restriction site does not change the amino acid coding sequence but introduces a restriction site for a tag to be optionally inserted. In some clones the nucleic acid molecule coding for the myc tag protein sequence (QKLISEEDL) was inserted immediately upstream of the newly inserted ECOR1 restriction site, i.e. in the region of the N terminus. The myc tag is recognised by a monoclonal antibody, which can be used in Western blot to identify the newly synthesised proteins. The nucleic acid molecule encoding the 1D4 tag was added immediately before the stop codon at the end of the NCKX5 sequence i.e. at the C terminus. The 1D4 tag is recognised by a monoclonal antibody, which can also be used in Western blot to identify the newly synthesised proteins.

The N-terminal sequence of hsNCKX2 from position 1 (M) to 120 (Q), plus a myc tag at position 84 of the WT sequence, was ligated to a partial construct of hsNCKX5 at amino acid 63 taking advantage of the EcoRI restriction site.

The sequences of the clones that were prepared as described above are given in 8 10 to 15.

### EXAMPLE 5

### Expression of NCKX5 in Host Cells

### Methods

Insect High Five™ cells were transfected with the vectors of Example 1e.g. coding for the full length human NCKX5 protein coupled to the leader sequence coding for the human NCKX2 signal peptide, using a suitable transfection method i.e. the use of the lepidopteron expression system method described by Farrell et al. (1998) Bio/Technology 60 pp 656-663.

High Five cells were collected and washed twice with 150 mM NaCl, 20 mM Hepes (pH 7.4), 80 mM sucrose, and 200 mM EDTA. The final pellets were resuspended in 200 ml of ice-cold radioimmune precipitation buffer containing 1% Triton X-100, 0.5% deoxycholate, 140 mM NaCl, 25 mM Tris (pH 7.5), 100 mM EDTA, and a protease inhibitor tablet (Roche Molecular Biochemicals catalogue number 1 836 170), and incubated on ice for 20 min. The samples were spun down in a microcentrifuge for 5 min at 20,000 3 g. Supernatants were removed and assayed for protein concentration using the Bradford dye-binding procedure (Bio-Rad). Bovine serum albumin was used as the standard in all protein assays.

Protein samples were separated on an 8% SDS-polyacrylamide gel and either stained with Gelcode Blue (Pierce) or transferred onto nitrocellulose (Bio-Rad) in 25 mM Tris buffer, pH 8.3, containing 192 mM glycine, 20% methanol, and 0.05% SDS. For Western blotting, the membranes were blocked for 1 h in TBST (10 mM Tris, pH 8.0, 100 mM NaCl, 0.05% Tween 20) and 10% skim milk, briefly rinsed in TBST, and subsequently incubated for 1 h at room temperature with primary antibody (1:20 dilution of PMe-1B3 or 10 mg/ml of 6H2 antibody) in TBST with 1% skim milk added. After washing, the membranes were incubated for 1 h with a 1:5000 dilution of sheep anti-mouse immunoglobulin conjugated to horseradish peroxidase (Amersham Pharmacia Biotech) in TBST plus 1% skim and then washed again. Immunodetection was carried out using LumiGlo chemiluminescent reagents (New England Biolabs).

HEK 293 cells were cultured in EMEM (Earle's minimum essential medium) at 37° C., 5% CO₂ in T175 cm² cell culture flasks containing sodium pyruvate and non-essential amino acids purchased from Biowhittaker and also containing 10% FCS (foetal calf serum) and 2 mM L-glutamine purchased from Sigma.

HEK293 cells were transfected with a vector as made in Example 2e.g. coding for the truncated human NCKX5 protein coupled to the leader sequence coding for the human NCKX2 signal peptide, using the calcium phosphate precipitation transfection method described by Kang et al (2005).

Results of the expression tests are shown in the Western blots of FIGS. 16 and 17. These blots show that the test cells expressed both NCKX5 and the NCKX2/NCKX5 chimera in their various tagged forms.

### EXAMPLE 6

### Analysis of Test Compounds Using NCKX5 Assay

### Materials

Earle's minimum essential medium (EMEM) containing sodium pyruvate and non-essential amino acids was purchased from Biowhittaker. Foetal calf serum (FCS), Dulbecco's modified essential medium (DMEM), L-glutamine, phosphate buffered saline (PBS), trypsin, dimethyl sulfoxide (DMSO) were purchased from Sigma. Fluo4-AM calcium sensitive dye was purchased from Molecular Probes. Glass bottomed 96 well plates were purchased from Greiner. FLEXstation and FLEXstation consumables were purchased from Molecular Devices.

### Cell Culture

HEK 293 cells were cultured in EMEM (Earle's minimum essential medium) at 37° C., 5% CO₂ in T175 cm² cell culture flasks containing sodium pyruvate and non-essential amino acids purchased from Biowhittaker and also containing 10% FCS (foetal calf serum) and 2 mM L-glutamine purchased from Sigma.

Transfection of HEK293 Cells with NCKX2/NCKX5 Chimera

HEK293 cells were transfected with a chimera coding for the full length human NCKX5 protein coupled to the leader sequence coding for the human NCKX2 signal peptide, in order to force protein expression at the plasma membrane, using a suitable transfection method, for example calcium phosphate precipitation as previously described (Kang et al (2005) J Biol Chem 280(8): 6823-6833.

Following transfection, HEK293 cells were plated into glass bottomed 96 well plates at an appropriate cell density, for example 2×10⁵ cells per well, and allowed to adhere overnight at 37° C., 5% CO₂.

### Assay Procedure

Adhered cells were washed with warmed PBS and incubated with up to 10 μM of a calcium-sensitive dye, Fluo4-AM dissolved in DMEM, for up to 1 h. Following dye loading, unhydrolysed dye was removed by washing cells twice with assay wash buffer as previously described (Kang et al 2005). Media was replaced with assay buffer depleted of Na+ to trigger reverse Ca²⁺/Na⁺ exchange (Kang et al 2005). Cells were then exposed to compounds from a compound library at various doses for various times at 37° C. Cells were transferred to a FLEXstation and treated with CaCl₂ followed by KCl to initiate potassium regulated Ca²⁺/Na⁺ exchange. Intracellular fluorescence was measured in real-time. At reaction plateau, saponin was added to cells to determine maximum fluorescence as a dye-loading control. Additionally, this method can be used to assess calcium or potassium dependence by addition of varying calcium or potassium concentrations added to test cells. This experiment may include a co-transfection with a fluorophor reporter plasmid to control for transfection efficiency.

For data analysis, the fluorescence of untreated blank cells is subtracted from test cells. The amount of calcium flux measured in unknown samples is quantitated by comparison to a serially-diluted reference standard curve. The effect(s) of test compounds on amount of calcium flux compared to untreated cells is determined and any increase or decrease in activity recorded. The assay can assess the effect of test compounds on maximal fluorescence, rate of fluorescence, Vₘₐₓ, Kₘ or time to maximum fluorescence compared to untreated cells. Test compounds determined to be exerting an effect can be re-tested over a broad concentration range to confirm efficacy.

### Automation of Assay

The assay can be performed manually but can also be automated, for example using a Hamilton robotic system. Once transfection and plating are complete, the robot is able to perform all washing, dye loading, temperature-dependent incubation of plates, compound additions and transfer of plates to FLEXstation. For higher throughput screening, the assay may be modified for use in 384 well or higher multi well plates.

### EXAMPLE 7

### Investigation of NCKX Activity in Mouse B16 Cells

All materials were purchased from Sigma except: B16 murine melanoma cells (ATCC); Fluo4-AM, Ribo Green (Molecular Probes); thapsigargin (Santa Cruz); Greiner black, glass-bottomed 96 well plates (Greiner Bio-One); Stealth™ RNAi duplexes, Lipofectamine 2000, Opti-MEM (Invitrogen); RNeasy total RNA extraction kits, QuantiTect PCR primers (Qiagen); first strand cDNA synthesis kits (Roche); ibidi μ-flow slides (ibidi Integrated Bio Diagnostics); SYBR Green PCR master mix (Bio-Rad);

### Cell Culture

B16 mouse melanoma cells were cultured in EMEM supplemented with 10% FCS and 2 mM L-glutamine at 37° C., 5% CO₂ in T175 cm² flasks and were sub-cultured twice weekly using trypsin-EDTA. HEK 293 cells were cultured in EMEM with 2 mM L-glutamine and Earle's BSS adjusted to contain 1.5 g/L sodium bicarbonate, 0.1 mM non-essential amino acids, and 1.0 mM sodium pyruvate, 90%; heat-inactivated horse serum, 10%

### Transfection of Stealth™ RNAi Duplexes Against Murine SLC24A5

24 h prior to transfection, B16 cells were plated at 25,000 cells/well in a 48-well plate. Cells in quadruplicate wells were transfected with 2 μg/ml Lipofectamine 2000 and 50 nM Stealth™ RNAi duplexes targeting SLC24A5 or scrambled controls (from Invitrogen). Reactions were also performed with Lipofectamine only or untreated negative controls. All dilutions were performed with Opti-MEM. RNAi duplex sequences were:

duplex 370
antisense: 3′-UGAAAGUUGCACCUGCAACAUCCUG-5′;
sense 5′-CAGGAUGUUGCAGGUGCAACUUUCA-3′;
duplex 370 scrambled control
antisense: 3′-UGACUAUUGACACGCGUCACAACUG-5′;
sense 5′-CAGUUGUGACGCGUGUCAAUAGUCA-3′;
duplex 762
antisense: 3′-UUCAAUUCUCUCCUCCAUAGCUCUG-5′
sense 5′-CAGAGCUAUGGAGGAGAGAAUUGAA-3′;
duplex 762 scrambled control
antisense: 3′-UUCUCACUUACUCUCCUCGAUACUG-5′;
sense 5′-CAGUAUCGAGGAGAGUAAGUGAGAA-3′
duplex 1265
antisense: 3′-AUAUCAAACACAUUGGAUCCCACGA-5′
sense 5′-UCGUGGGAUCCAAUGUGUUUGAUAU-3′;
duplex 256
antisense 3′-UAAUGAGGAAGUAGAUUACGAUACC-5′
sense 5′-GGUAUCGUAAUCUACUUCCUCAUUA-3′;
duplex 567
antisense 3′-AUACACUGCACAGUCUCGGAAGAGG-5′
sense 5′-CCUCUUCCGAGACUGUGCAGUGUAU-3′.

Cells were incubated with transfection reagents for 6-8 h, reagents were then removed and replaced with phenol red-free DMEM supplemented with 10% FCS and 4 mM L-glutamine for 72 h. At 72 h, cells were taken for viability and total protein determination and supernatants analysed for melanin production.

SLC8 and SLC24 mRNA Analysis B16 Murine Melanoma Cells

Following trypsinisation, total RNA was extracted from B16 cells using the RNeasy mini kit, with on-column DNAse treatment, as per the manufacturer's instructions. Total RNA concentration of extracts was quantified using Ribo Green as per the manufacturer's instructions, with fluorescence measured with a BMG Fluostar Optima plate reader. Absorbance values for unknown samples were compared to a seven point reference curve with a dynamic range of 15 ng/μl-1 μg/μl.

First strand cDNA synthesis was performed from 1 μg total RNA by reverse transcription using the first strand synthesis kit, as per the manufacturer's instructions. RT was performed in 20 μl reactions and where more than 20 μl cDNA was required, multiple reactions were performed and cDNA pooled. SLC24 mRNA expression was then analysed by real-time PCR with SYBR Green detection using a Bio-Rad icycler. QuantiTect PCR primers directed against exon-exon boundaries within murine SLC8A1-SLC8A3, murine SLC24A1-SLC24A6 and murine GAPDH were purchased from Qiagen. Primer efficiency was confirmed by serial dilution of cDNA and melt-curve analysis. Target gene expression was normalised against GAPDH mRNA expression using the Δ_{CT} method.

See table of FIG. 22 showing that NCKX5 (SLC24A5) is expressed in B16 cells but that SLC8 is not.

SLC8 and SLC24 mRNA Analysis Dark Human Melanocytes

Following trypsinisation, total RNA was extracted from dark human melanocytes using the RNeasy mini kit, with on-column DNAse treatment, as per the manufacturer's instructions. Total RNA concentration of extracts was quantified using Ribo Green as per the manufacturer's instructions, with fluorescence measured with a BMG Fluostar Optima plate reader. Absorbance values for unknown samples were compared to a seven point reference curve with a dynamic range of 15 ng/μl-1 μg/μl.

First strand cDNA synthesis was performed from 1 μg total RNA by reverse transcription using the first strand synthesis kit, as per the manufacturer's instructions. RT was performed in 20 μl reactions and where more than 20 μl cDNA was required, multiple reactions were performed and cDNA pooled. SLC24 mRNA expression was then analysed by real-time PCR with SYBR Green detection using a Bio-Rad icycler. Primer sequences were:

SLC24A1:
forward: 5′-TCTGCACAACAGCACCAT-3′;
reverse 5′-CTCTCCTCCTCCTTCTCCTT-3′;
SLC24A2:
forward: 5′-ATGATACACACCCTTGACC-3′;
reverse 5′-CCTTTTCTCTGAACCTCCCTT-3′;
SLC24A3:
forward: 5′-CGTCTTATACTTCACTGTACCC-3′;
reverse 5′-AACCAATGATTGTGACCATCC-3′;
SLC24A4:
forward: 5′-GACACAGACAGCCAAGAA-3′;
reverse 5′-GCATAGAACATATACAGAGCACCA-3′;
SLC24A5:
forward: 5′-GAGATGGAGGCATCATAATCTA-3′;
reverse 5′-CCTGAGACAATCCAAGGGATTC-3′
SLC24A6:
forward: 5′-AGGCTTCACTGGCTCTT-3′;
reverse 5′-AGGCATCTCCAATGCTGTTC-3′;
GAPDH:
forward: 5′-GGACCTGACCTGCCGTCT-3′;
reverse: 5′-TAGCCCAGGATGCCCTTG-3′.

QuantiTect PCR primers directed against exon-exon boundaries within human SLC8A1-SLC8A3 were purchased from Qiagen. Primer efficiency was confirmed by serial dilution of cDNA and melt-curve analysis. Target gene expression was normalised against GAPDH mRNA expression using the Δ_{CT} method.

### Analysis of Intracellular NCKX Activity in Various Cells in Suspension

An assay for intracellular Na-induced Ca²⁺ release was developed based on that previously described (Altimimi & Schnetkamp 2007). B16 cells were trypsinised and centrifuged at 300 g, 3 min. Cells were resuspended with 500 μl basal DMEM and 12 μM Fluo3-AM and incubated for 35 min at room temperature with rotation on an orbital rotary mixer. Cells were centrifuged 300 g, 2 min and washed in 1.5 ml Na⁺ loading media. Cells were resuspended in 275 μl Na⁺-loading buffer (150 mM NaCl, 20 mM Hepes pH 7.4 with arginine, 3 mM KCl, 1.5 mM CaCl₂, 10 mM glucose, 250 μM sulfinpyrazone). 50 μl B16 cells were suspended in 2 ml KCl media in a plastic, clear on all sides, cuvette. A magnetic flea was placed in the cuvette and the cuvette placed in a luminescence spectrometer with magnetic stirrer enabled and jacket heated to 25° C. The trace was started with readings every 1 second. At 10 seconds 2 μM FCCP, 1 μM gramicidin and 1 μM thapsigargin was added. At 180 seconds, 75 mM NaCl was added, at 200 seconds 350 μM CaCl₂ was added and once the trace had reached plateau, 0.01% saponin was added. The mean fluorescence counts of the 10 seconds immediately prior to sodium addition were used to subtract background signal from each of the data points of the sodium-induced calcium trace. The mean fluorescence counts for the final 10 seconds of the saponin-induced trace were then used to normalise each data point of the sodium-induced calcium trace. FIG. 18 shows the dose-dependent nature of Na+ dependent Ca2+ release in B16 cells when studied in cuvette suspension.

### Analysis of Intracellular NCKX Activity in Various Cells by HTS Method

The suspension assay previously described was modified for use in 96 well formats (Altimimi & Schnetkamp 2007). Cells were plated into Greiner glass bottomed 96 well plates at 100,000 cells/well and adhered overnight. Media was removed and cells loaded with the Ca²⁺-sensitive dye Fluo4-AM in serum-free media at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing, 30 μl sodium-loading buffer placed onto cells. 100 μl of KCl assay buffer (150 mM KCl, 20 mM Hepes pH 7.4 with arginine, 100 μM EDTA) containing 2 μM FCCP, 1 μM gramicidin and 1 μM thapsigargin was placed onto cells and the plate inserted into a Molecular Devices FLEXStation. Fluorescence readings were taken every 3 seconds for 300 seconds. 120 mM NaCl was added at 120 seconds, 350 μM CaCl₂ was added at 200 seconds and 0.01% saponin added at 260 seconds. Following analysis, data was transferred to Excel. Background fluorescence was subtracted from each Na⁺ and saponin-induced fluorescence components. Background subtracted Na⁺ fluorescence was then normalised to background subtracted saponin fluorescence by multiplication of Na⁺ data by the reciprocal of saponin data.

The suitability of this assay for HTS was assessed by analysis of the Z′ factor, signal to noise ratio and signal to background ratio. One plate of B16 cells was analysed on each of three separate days as described above. The Z′ factor was calculated as: {1−[(3*agonist SD)+(3*NSB SD)]}/(agonist mean−NSB mean) as previously described (Chen 2006, Zhang 1999) where NSB is non-specific background. The Z′ factor for this assay was 0.4, S:N 22.5, S:B 2.8, suggesting that the assay was suitable for HTS. This assay format was automated for use on a Hamilton robotic platform for HTS.

FIGS. 19 and 20 show the does dependent nature of Na+ dependent Ca2+ release in B16 cells when studied in 96 well assays that simulate high throughput assays.

### Analysis of Intracellular NCKX Activity in Various Cells by Real-Time Confocal Microscopy

The assay described was adapted for use by real-time confocal microscopy on a Leica TCS SP1 confocal microscope. Cells were seeded into ibidi μ-flow slides and adhered overnight. Media was removed and cells loaded with Fluo4-AM in serum-free DMEM at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing the slide was placed on the stage of the Leica TCS SP1 Confocal Scanning Laser Microscope (Leica Microsystems GmbH, Wetzlar, Germany). The scanning head was fitted to an inverted Leica DM IRBE microscope. A 40X plan apo 1.25 n.a. oil immersion phase contrast objective was used for collecting the images simultaneously in fluorescence and phase contrast. For image acquisition a frame size of 512×512 pixels was chosen and the sample scan rate was set to either maximum (1 frame every 0.87 seconds) or more typically one frame every 2 seconds. An argon ion laser with an excitation wavelength of 488 nm was used to excite the Fluo-4-loaded cells. Fluorescence emission was captured from 500-585 nm. The field of view in all images was 250×250 μm. Typical datasets were collected over 5 minutes. At time zero the cells were switched to KCl assay buffer (150 mM KCl, 20 mM Hepes pH 7.4 with arginine, 100 μM EDTA containing 2 μM FCCP, 1 μM gramicidin and 1 μM thapsigargin). At 180 seconds, cells were switched to 75 mM NaCl and at 250 seconds cells were switched to 350 μM CaCl₂ buffer. Analysis of the images and the export of .avi movie files were performed via Leica LCS operating software and the results exported as .xml files. Pixel intensity values over time were assessed by highlighting cells within the image and plotting these as a function of time.

FIG. 21 demonstrates the confocal microscopy approach showing Na+ dependent Ca2+ release in B16 cells.

### Analysis of Heterologous NCKX2 Activity in HEK 293 Cells

Cultured HEK 293 cells were plated at 10,000 cells per well in glass bottomed 96 well plates and adhered overnight. Following adherence, cells were transfected with the short splice variant of the hNCKX2 gene cloned into pcDNA3.1 expression vector. The short splice variant lacks a stretch of 17 amino acids within the cytoplasmic loop of the protein. A c-Myc tag was also inserted at the BstE II site between bases 241-242, corresponding to amino acid residue 81 (Prinsen et al 2000, Winkfein et al 2003). Transfections were performed with Mirus 293 reagent as per the manufacturer's instructions for 48 h. Following transfection, media was removed and cells loaded with Fluo4-AM in serum-free DMEM at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing, 30 μl sodium-loading buffer placed onto cells. 100 μl of KCl assay buffer (150 mM KCl, 20 mM Hepes pH 7.4 with arginine, 100 μM EDTA) containing 2 μM FCCP, 1 μM gramicidin and 1 μM thapsigargin was placed onto cells and the plate inserted into a Molecular Devices FLEXStation. Fluorescence readings were taken every 3 seconds for 500 seconds. 350 μM CaCl₂ was added at 150 seconds, 75 mM NaCl was added at 180 seconds and 0.01% saponin added at 350 seconds. Alternatively, HEK 293 cells heterologously expressing hNHCX2 were loaded with Fluo4-AM for 30 min at 37° C. in a physiological salt solution (150 mM NaCl, 20 mM HEPES pH 7.4 with arginine, 6 mM glucose, 0.25 mM sulfinpyrazone, 0.1 μM ouabain ±1.5 mM CaCl₂). Following removal of extracellular dye, cells were switched to LiCl₂ assay buffer (150 mM LiCl₂, 20 mM HEPES pH 7.4 with arginine, 6 mM glucose, 0.1 mM EDTA). At 120 seconds, 350 μM CaCl₂ was added followed by 50 mM KCl to initiate maximal rate of reverse calcium exchange. At maximal fluorescence 0.01% saponin was added. Following analysis, data was transferred to Excel. Background fluorescence was subtracted from each Na⁺ and saponin-induced fluorescence components. Background subtracted Na⁺ fluorescence was then normalised to background subtracted saponin fluorescence by multiplication of Na⁺ data by the reciprocal of saponin data. See FIG. 23.

### REFS FOR EXAMPLE 7

Altimimi H. F, Schnetkamp P. P. M. (2007), J Biol Chem 282(6): 3720-3729
Chen C, Smith C, Minor L, Damiano B. Development of FLIPR-based HTS Assay for Gi-Coupled GPCRs. In Handbook of Assay Development in Drug Discovery, pages 305-317. Published by Taylor Francis. 2006.
Zhang J H, Chung T D, Oldenburg K R 1999. A Simple Statistical Parameter for Use in Evaluation and Validation of High Throughput Screening Assays. J Biomol Screen 4(2): 67-73.
Prinsen C F, Szerencsei R T, Schnetkamp P P. Molecular cloning and functional expression of the potassium-dependent sodium-calcium exchanger from human and chicken retinal cone photoreceptors. J Neurosci 2000; 20:1424-1434.
Winkfein R J, Szerencsei R T, Kinjo T G, Kang K, Perizzolo M, Eisner L, Schnetkamp
P P. Scanning mutagenesis of the alpha repeats and of the transmembrane acidic residues of the human retinal cone Na⁺/Ca²⁺—K⁺ exchanger. Biochemistry 2003; 42:543-552.

### EXAMPLE 8

### Methods for the Assessment of NCKX5 Activity in Relation to Melanogenesis in B16 Cells

### A) Quantification of Melanin Production, Protein Content and Cell Viability of Cultured B16 Mouse Melanocytes.

### 1) Cell Treatments 72 Hours Post-Knockdown:

The phenol free DMEM culture media was removed from each well for quantification of secreted melanin. Each well of the culture plate was then rinsed once with Dulbeccos phosphate buffered saline (dPBS) (Sigma-Aldrich, Poole, UK) and replaced with 0.5 ml Wst1 reagent (diluted 1/10 in media) (Roche Diagnostics Ltd, West Sussex, UK). The culture plate was incubated at 37° C., 5% CO₂ for 30 minutes. The Wst1 reagent was removed from each well for determination of cell viability and each well was again rinsed with dPBS. 200 μl of 1% w/v Triton X-100 (Sigma-Aldrich, Poole, UK) in dPBS was added to each well of the culture plate. The plate was incubated for 20 minutes at 4° C. on an orbital shaker. Supernatant from each well was removed and centrifuged at 13000 g for 5 minutes to remove cell debris. Soluble fractions were kept for protein quantification.

### 2) Melanin Assay:

1 mg synthetic melanin (Sigma-Aldrich, Poole, UK) was dissolved in 50 μl of 100% DMSO. 950 μl phenol free DMEM+10% FCS was added drop-wise to create a 1 mg/ml top standard. This standard was serially diluted (5-fold) in media+FCS to give a standard curve for quantification of melanin in sample preparations (range from 1000 to 8 μg/ml). 100 ul each standard and sample was placed in a 96-well microtitre plate in duplicate (Greiner Bio-One Ltd, Gloucestershire, UK) and the optical density (OD) of each sample replicate was measured at 450 nm using a Dynex MRX plate reader (Dynex Technologies Ltd, West Sussex, UK). The melanin content of each culture fraction was calculated from the synthetic melanin standard curve.

### 3) Wst1 Assay:

The OD⁴⁵⁰ of 100 μl fractions (measured in duplicate) of post-incubation Wst1 reagent was measured. The OD⁴⁵⁰ of each treatment was compared to evaluate relative viability.

### 4) Protein Content Determination:

The protein content of each triton X-100 cell culture fraction was measured using a BCA assay kit (Perbio Science UK Ltd, Northumberland, UK) as per the manufacturers' instructions with the following modification: −10 μl of each solubilised protein fraction was placed in a 96-well microtitre plate in duplicate. 15 μl dH2O was added to each sample prior to the addition of the BCA colour reagent. A standard curve was prepared by 2-fold serial dilution (using 1% triton X-100 in dPBS) of BSA (range=2000 to 15.6 μg/ml). The plate containing all samples and standard solutions was incubated for 15 minutes and the OD⁵⁹⁵ was measured. Protein content of each sample was calculated from the BSA standard curve.

### 5) Calculation of μg Melanin Per μg Protein:

The μg/ml melanin content for each sample was divided by 2 to give the total melanin per sample. The μg/ml protein content derived from the BCA assay was multiplied by 2.5 (to account for the assay dilution step, then divided by 5 (accounting for the volume of triton used to lyse the cells and release protein. Finally, for each treatment, the total melanin (μg) value was divided by the total protein (μg) value to give μg melanin per μg protein.
See FIG. 29 for results showing that NCKX5 protein does modulate pigment production.

### B) Quantification of Melanin Production, NCKX5 Protein Expression and Cell Viability of Cultured Human Melanocytes.

After siRNA-mediated knockdown of SLC24A5, the following assays were conducted:

### 1) Cell Viability Assessment:

Melanocyte viability 5 days after siRNA treatment was assessed by Wst1 assay as described in A) but with the following modifications: 1 ml of 1/10 diluted (in culture media) Wst1 reagent was added to each well of 6-well culture plates containing human melanocytes. The plates were incubated for 60 minutes at 37° C., 5% CO² prior to the removal and OD⁴⁵⁰ assessment of the reagent.

### 2) Protein and Melanin Fractionation:

Cultured Cells (trypsinised off of 6-well culture plates) were lysed on ice for 20 minutes in 1.5 ml eppendorfs using 100 μl per sample of 1% triton X-100 in dPBS containing protease inhibitor cocktail (Sigma-Aldrich, Poole, UK). Cell extracts were centrifuged (10 minutes at 13000 g) to separate melanin and cell debris from the solubilised protein. The protein concentration of each supernatant fraction was determined by BCA assay as described in A).

### 3) SDS-Page and Electrophoretic Transfer to PVDF Membranes:

20 μg protein (as determined by BCA assay) was reconstituted into 20 μl of 1×LDS loading buffer (Invitrogen Ltd, Paisley, UK) containing 1× reducing agent (Invitrogen Ltd, Paisley, UK), heated to 40° C. for 30 minutes and loaded onto 10% Novex bis-tris acrylamide gels with 1×MOPS running buffer (Invitrogen Ltd, Paisley, UK). Kaleidoscope molecular weight markers (Bio-Rad Laboratories Ltd, Hemel Hempstead, UK) were run alongside samples for size determinations. Protein was transferred onto PVDF membrane by electrophoresis transfer, using a Bio-Rad mini-cell II trans-blotter and 1× Tris-Glycine transfer buffer (Invitrogen Ltd, Paisley, UK)+15% v/v methanol (100V for 1 hour).

### 4) Western Blotting to Detect NCKX5:

Membranes containing transferred protein were “blocked” using 2% w/v skimmed milk protein (SMP) in PBS+0.05% tween20 (PBST) for 1 hour with gentle agitation. Peptide affinity purified rabbit polyclonal antibody (raised using a peptide equivalent to the C-terminus of NCKX5-GNNKIRGCGG) was diluted to 0.5 μg/ml using 2% SMP in PBST. The diluted antibody was incubated with the membrane for 2 hours at room temperature. The membrane was then rinsed with PBST (4×5 minutes washes). Peroxidase conjugated anti-rabbit IgG (Jackson ImmunoResearch Laboratories Inc. PA, USA) was diluted 1/4000 using 2% SMP in PBST and was incubated with the membranes for 1 hour. Finally, membranes were washed using 6×5 minute rinses with PBST. SuperSignal Western pico (Perbio Science UK Ltd, Northumberland, UK) chemiluminescence detection reagents was used to probe the membranes for secondary antibody binding as per the manufacturers instructions. Visualisation of results was via a Chemidoc XRS imaging system (Bio-Rad Laboratories Ltd, Hemel Hempstead, UK).

### 5) Measurement of Melanin Content:

To each pellet of melanin+cell debris obtained in section 2), 0.5 ml of diethyl ether:ethanol (1:1 ratio) was added. Eppendorfs were vortexed vigorously for 30 seconds and centrifuged at 13000 g for 5 minutes. The ether layer was carefully removed to waste and a further 0.5 ml was added. The tubes were agitated and centrifuged as before. The liquid phase was again removed and the melanin pellets were allowed to air dry at room temperature for 10 minutes. Melanin pellets were solubilised by the addition of 200 μl of 1M NaOH+10% DMSO and incubation (with occasional agitation) at 50° C. for 1 hour. 80 μl aliquots of each sample were transferred to a 96-well microtitre plate in duplicate. OD⁴⁵ 0 of each sample was determined and the melanin content of each fraction was calculated from a synthetic melanin standard curve, prepared as described above. In treatment comparisons, results were expressed as μg melanin per μg protein.

See FIGS. 30 and 31 for results showing siRNA knockdown demonstrating a reduction of SLC24A5 mRNA in mouse B16 cells and protein content results.

### EXAMPLE 9

### sIRNA Knockdown Results in Human Cells

### Cell Culture

Primary human melanocytes isolated from lightly pigmented or darkly pigmented neonatal foreskin were obtained from Cascade Biologics. Melanocyte Growth Medium (MGM) refers to Medium 254 (Cascade Biologics) supplemented with HMGS (Cascade Biologics). Melanocyte cultures were maintained in MGM at 37° C. with 10% CO₂. Cells were seeded at either 2×10⁴ cells/cm² (melanogenesis experiments) or 1×10⁴ cells/cm² (immunofluorsecence experiments) and allowed to attach for 24 h prior to transfection.

Transfection of Cells with Oligonucleotides

Stealth™ siRNA duplex oligonucleotides (Table 1) were purchased from Invitrogen and used at a final concentration of 20-100 nM. A scrambled, non-targeting version of duplex 260 was used as a control.

TABLE

Sequences and exon targets of siRNA duplexes
designed against human SLC24A5

| | | |
|---|---|---|
| siRNA | | Exon |
| duplex | Sequence | target |

| | | |
|---|---|---|
| 185 | 5′ AGGGCCACAGGAAATAGCACCCAAT 3′ | 1 & 2 |
| | 3′ TCCCGGTGTCCTTTATCGTGGGTTA 5′ | boundary |
| 260 | 5′ GAGCGCAGAGATGGAGGCATCATAA 3′ | exon 2 |
| | 3′ CTCGCGTCTCTACCTCCGTAGTATT 5′ | |
| 301 | 5′ CGTTTACATGTTCATGGCCATATCT 3′ | exon 2 |
| | 3′ GCAAATGTACAAGTACCGGTATAGA 5′ | |
| 492 | 5′ GCACCATCCTTGGATCTGCAATTTA 3′ | 3 & 4 |
| | 3′ CGTGGTAGGAACCTAGACGTTAAAT 5′ | boundary |
| 1110 | 5′ CCGCATTTACATATATCCTGGTTTG 3′ | exon 7 |
| | 3′ GGCGTAAATGTATATAGGACCAAAC 5′ | |

Lipofectamine™ 2000 (Invitrogen) was diluted (1:50) in Opti-MEM® I Reduced Serum Medium (Gibco) and incubated at room temperature for 15 min. siRNA duplexes were diluted in Opti-MEM® I and combined with 1 volume of diluted Lipofectamine™ 2000. Following a 15 min incubation period at room temperature, 4 volumes of Opti-MEM® I was added, and the resultant mixture used to transfect the cells. Following a 6-8 h incubation period at 37° C. with 10% CO₂, the cells were transferred to MGM. Cells were transfected on day 0 and again on day 5 if required.

### Immunofluorescence

Cells were grown on glass coverslips and transfected with siRNA duplexes where indicated. Following a 72 h, 5 day or 10 day incubation, cells on coverslips were washed twice with PBS then fixed with 2% PFA in PBS for 20 min, washed a further 3 times, and permeabilised with 0.5% saponin in PBS. Coverslips were incubated with 0.2% BSA/0.1% saponin/PBS for 1 h at room temperature, then incubated with primary antibodies in 0.2% BSA/0.1% saponin/PBS for 1.5 h at room temperature. Primary antibody dilutions used were anti-NCKX5(827), 1:500; anti-NCKX5(826), 1:25 and anti-TGN46 (AbD Serotec), 1:200. Coverslips were washed twice with 0.1% saponin/PBS and twice with 0.2% BSA/0.1% saponin/PBS, then incubated with secondary antibodies in 0.2% BSA/0.1% saponin/PBS for 45 min at room temperature. Secondary antibodies (Alexa 488-conjugated anti-rabbit or Alexa 633-conjugated anti-sheep) were purchased from Invitrogen and used at a dilution of 1:500. Coverslips were washed twice with 0.2% BSA/0.1% saponin/PBS and twice with 0.1% saponin/PBS, then rinsed with Milli Q and mounted with VectaShield (Vector Laboratories) mounting medium. Cells were observed and photographed using a confocal microscope.

See FIG. 24 for results of SLC24A5 reduction over time by siRNA duplexes. See FIG. 25 for visual interpretation of the reduction of melanin pigment in human melanocytes after knockdown when compared to non-knockdown results. See FIG. 25 for Immunofluorescence results. And also see FIG. 27 for quantitative analysis results showing melanin production reduced in knowncdown siRNA treated cells.

### EXAMPLE 10

### Demonstration of Lack of Native SLC24A5 (NCKX5) in HEK 293 Cells

### Sodium-Induced Intracellular Calcium Release in Various Cells

We have demonstrated that SLC24A5 mRNA is undetectable in HEK 293 cells and human keratinocytes. Moreover, transcript expression is lower in MEWO cells than primary human melanocytes or B16 cells. Therefore we investigated the extent of Na+-induced intracellular Ca²⁺ release in these cells. 100,000 cells were plated in triplicate to a 96 well plate, adhered overnight and intracellular Ca²⁺ release investigated as described in “Analysis of intracellular NCKX activity in various cells by HTS method”. Whilst some activity was detected in HEK 293, MEWO and keratinocytes, the rank order of normalised activity is follows our prediction based on transcript expression profiles (FIG. 32).

The early time-points from the trace on FIG. 33 show a clear difference in rates of sodium-induced calcium release from an intra-cellular store between cells expressing high levels of SLC24A5 transcript (dark and light melanocytes, B16 melanocytes) and those with undetectable SLC24A5 transcript (HEK-293 and ketratinocytes). The MeWo cells have intermediate levels of SLC24A5 transcript (and do not produce melanin under these conditions) which corresponds with an intermediate level of sodium-induced calcium release.

SLC24 and SLC8 mRNA Expression HEK 293 and Human Keratinocytes

To assess if SLC24A5 and SLC8 family members are expressed at the transcript level in HEK 293 cells, RNA was extracted from cultured HEK 293 cells and real-time PCR performed. SLC24A5 mRNA was undetected and SLC8 mRNA was detected at low levels. Previous studies have demonstrated that plasma membrane NCKX activity is undetected in untransfected HEK 293 cells suggesting that NCKX1-NCKX4 proteins are not expressed at detectable levels (Kang et al 2005; Cooper et al 1999; Visser et al 2007).

| Gene | C_{T} ± SD |
|---|---|
| SLC24A5 | Not detected |
| SLC8A1 | 31.7 ± 0.2 |
| SLC8A2 | 30.6 ± 0.2 |
| SLC8A3 | 31.5 ± 0.2 |

Table above shows 1 μg RNA extracted from HEK293 cells was reverse-transcribed and real-time PCR performed as described in methods. Data are mean±SD triplicate reactions.

| Gene | C_{T} ± SD |
|---|---|
| SLC24A1 | 27.4 ± 0.2 |
| SLC24A2 | 36.1 ± 0.5 |
| SLC24A3 | 29.8 ± 0.0 |
| SLC24A4 | Not detected |
| SLC24A5 | 34.2 ± 0.6 |
| SLC24A6 | 24.0 ± 0.0 |
| SLC8A1 | 31.8 ± 0.2 |
| SLC8A2 | 33.8 ± 0.3 |
| SLC8A3 | Not detected |
| SNARE | 21.2 ± 0.1 |

Table above shows mRNA extracted from human keratinocytes detected using SYBR green realtime PCR following reverse transcription of 1 μg of RNA. Table values are mean±SD duplicate reactions.

### REFS FOR EXAMPLE 10

K J Kang, T G Kinjo, R T Szerencsei, P P M Schnetkamp (2005). Residues Contributing to the Calcium and Potassium Binding Pocket of the NCKX2 Na⁺/Ca²⁺—K⁺ Exchanger. J Biol Chem 280(8): 6823-6833.
C B Cooper, R J Winkfein, R T Szerencsei, P P M Schnetkamp (1999). cDNA Cloning and Functional Expression of the Dolphin Retinal Sodium-calcium-Potassium Exchanger NCXK1: Comparison with the Functionally Silent Bovine NCKX1. Biochemistry 38: 6276-6283.
F Visser, V Valsecchi, L Annunziato, J Lytton (2007). Exchangers NCKX2, NCKX3, and NCKX4: Identification of Thr-551 as a Key Residue in Defining the Apparent K⁺ Affinity of NCKX2. J Biol Chem 282(7): 4453-62.

### EXAMPLE 11

### Pharmaceutical and Cosmetic Formulations

A further aspect of the invention provides a pharmaceutical formulation comprising a compound isolated in step (i) of the method of the first aspect of the invention in admixture with a cosmetically, pharmaceutically or veterinarily acceptable adjuvant, diluent or carrier.

Preferably, the formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered topically, orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for example, the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that the overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

### Dermatologically Acceptable Vehicle

The composition used according to the invention also comprises a dermatologically/cosmetically acceptable vehicle to act as a dilutant, dispersant or carrier for the actives. The vehicle may comprise materials commonly employed in skin care products such as water, liquid or solid emollients, silicone oils, emulsifiers, solvents, humectants, thickeners, powders, propellants and the like.

The vehicle will usually form from 5% to 99.9%, preferably from 25% to 80% by weight of the composition, and can, in the absence of other cosmetic adjuncts, form the balance of the composition.

### Optional Skin Benefit Materials and Cosmetic Adjuncts

Besides the actives, other specific skin-benefit actives such as sunscreens, skin-lightening agents, skin tanning agents may also be included. The vehicle may also further include adjuncts such as antioxidants, perfumes, opacifiers, preservatives, colourants and buffers.

### Product Preparation, Form, Use and Packaging

To prepare the topical composition used in the method of the present invention, the usual manner for preparing skin care products may be employed. The active components are generally incorporated in a dermatologically/cosmetically acceptable carrier in conventional manner. The active components can suitably first be dissolved or dispersed in a portion of the water or another solvent or liquid to be incorporated in the composition. The preferred compositions are oil-in-water or water-in-oil or water-in-oil-in-water emulsions.

The composition may be in the form of conventional skin-care products such as a cream, gel or lotion, capsules or the like. The composition can also be in the form of a so-called “wash-off” product e.g. a bath or shower gel, possibly containing a delivery system for the actives to promote adherence to the skin during rinsing. Most preferably the product is a “leave-on” product; a product to be applied to the skin without a deliberate rinsing step soon after its application to the skin.

The composition may packaged in any suitable manner such as in a jar, a bottle, tube, roll-ball, or the like, in the conventional manner. It is also envisaged that the inventive compositions could be packaged as a kit of two separate compositions one containing the petroselinic acid and the second containing the phenolic compound, to be applied to the skin simultaneously or consecutively.

The composition according to the invention may also be formulated into a form suitable for oral ingestion such as a capsule, tablet or similar.

The method of the present invention may be carried out one or more times daily to the skin which requires treatment. The improvement in skin appearance will usually become visible after 3 to 6 months, depending on skin condition, the concentration of the active components used in the inventive method, the amount of composition used and the frequency with which it is applied. In general, a small quantity of the composition, for example from 0.1 to 5 ml is applied to the skin from a suitable container or applicator and spread over and/or rubbed into the skin using the hands or fingers or a suitable device. A rinsing step may optionally follow depending on whether the composition is formulated as a “leave-on” or a “rinse-off” product.

The formulation below describes an oil in water cream suitable for the methods and uses according to the present invention. The percentages indicated are by weight of the composition.

| | wt % | Wt % | Wt % |
|---|---|---|---|
| Mineral Oil | 4 | 4 | 4 |
| Petroselinic acid | 1.15 | 2 | 3 |
| (triglyceride) ex Elysion | | | |
| Green Tea Polyphenols | 0 | 2 | 0 |
| EGCG | 0 | 0 | 1 |
| Quercetin | 0.5 | 0 | 0 |
| Brij 56* | 4 | 4 | 4 |
| Alfol 16RD* | 4 | 4 | 4 |
| Triethanolamine | 0.75 | 0.75 | 0.75 |
| Butane-1,3-diol | 3 | 3 | 3 |
| Xanthan gum | 0.3 | 0.3 | 0.3 |
| Perfume | qs | qs | qs |
| Butylated hydroxy toluene | 0.01 | 0.01 | 0.01 |
| Water | to 100 | to 100 | to 100 |

*Brij 56 is cetyl alcohol POE (10)
Alfol 16RD is cetyl alcohol

The formulation below describes an emulsion cream according to the present invention.

| FULL CHEMICAL | | | | |
|---|---|---|---|---|
| NAME OR CTFA NAME | TRADE NAME | WT. % | WT. % | WT % |
| Coriander seed oil ex | | 2.0 | 3 | 1.5 |
| Loders Croklaan (PA | | | | |
| triglyceride 60-75% | | | | |
| of total fatty acids) | | | | |
| Gallic acid | | 1 | 0 | 0 |
| Genistein | | 0 | 2 | |
| Diadzein | | 0 | 0 | 1.5 |
| Disodium EDTA | Sequesterene Na2 | 0.05 | 0.05 | 0.05 |
| Magnesium aluminium | Veegum Ultra | 0.6 | 0.6 | 0.6 |
| silicate | | | | |
| Methyl paraben | Methyl Paraben | 0.15 | 0.15 | 0.15 |
| Simethicone | DC Antifoam | 0.01 | 0.01 | 0.01 |
| | Emulsion | | | |
| Butylene glycol 1,3 | Butylene Glycol 1,3 | 3.0 | 3.0 | 3.0 |
| Hydroxyethylcellulose | Natrosol 250HHR | 0.5 | 0.5 | 0.5 |
| Glycerine, USP | Glycerine USP | 2.0 | 2.0 | 2.0 |
| Xanthan gum | Keltrol 1000 | 0.2 | 0.2 | 0.2 |
| Triethanolamine | Triethanolamine (99%) | 1.2 | 1.2 | 1.2 |
| Stearic acid | Pristerene 4911 | 3.0 | 3.0 | 3.0 |
| Propyl paraben NF | Propylparaben NF | 0.1 | 0.1 | 0.1 |
| Glyceryl | Naturechem GMHS | 1.5 | 1.5 | 1.5 |
| hydrostearate | | | | |
| Stearyl alcohol | Lanette 18 DEO | 1.5 | 1.5 | 1.5 |
| Isostearyl palmitate | Protachem ISP | 6.0 | 6.0 | 6.0 |
| C12-15 alcohols | Hetester FAO | 3.0 | 3.0 | 3.0 |
| octanoate | | | | |
| Dimethicone | Silicone Fluid | 1.0 | 1.0 | 1.0 |
| | 200 (50 cts) | | | |
| Cholesterol NF | Cholesterol NF | 0.5 | 0.5 | 0.5 |
| Sorbitan stearate | Sorbitan Stearate | 1.0 | 1.0 | 1.0 |
| Butylated | Embanox BHT | 0.05 | 0.05 | 0.05 |
| hydroxytoluene | | | | |
| Tocopheryl acetate | Vitamin E Acetate | 0.1 | 0.1 | 0.1 |
| PEG-100 stearate | Myrj 59 | 2.0 | 2.0 | 2.0 |
| Sodium stearoyl | Pationic SSL | 0.5 | 0.5 | 0.5 |
| lactylate | | | | |
| Hydroxycaprylic acid | Hydroxycaprylic Acid | 0.1 | 0.1 | 0.1 |
| Alpha-bisabolol | Alpha-bisabolol | 0.2 | 0.2 | 0.2 |
| Water, DI | | q.s. to 100 | q.s. to 100 | q.s. to 100 |

Both the above topical compositions of the above formulations provide an effective cosmetic treatment to improve the appearance of wrinkled, aged, photodamaged, and/or irritated skin, when applied to normal skin that has deteriorated through the aging or photoageing or when applied to youthful skin to help prevent or delay such deteriorative changes. The compositions are also effective for soothing irritated skin, conditioning dry skin, lightening skin colour and reducing oil and sebum secretions. The compositions can be processed in conventional manner.

1. A method of identifying compounds that either increase or decrease skin and/or hair pigmentation, or alter the melanin composition of skin and/or hair, the method comprising determining the ability of a test compound to modulate NCKX-mediated calcium ion movement across a membrane.

2. A method as claimed in claim 1 comprising the steps of exposing a membrane comprising a NCKX molecule or variant, fusion or derivative thereof to a test compound and measuring either directly or indirectly the calcium ion concentration on one or both sides of the membrane.

3. A method as claimed in claim 2 comprising the steps of:
(a) providing a membrane comprising at least one NCKX molecule or functionally equivalent variants, fusions or derivatives thereof, wherein said membrane separates two distinct compartments;
(b) measuring the Calcium ion (Ca²⁺) concentration in both compartments before exposure to one or more test compounds;
(c) exposing the membrane to one or more test compounds;
(d) measuring the Calcium ion (Ca²⁺) concentration in both compartments after exposure to one or more test compounds;
(e) identifying the amount of Calcium ion (Ca²⁺) movement across the membrane by comparing the concentrations measured in step (b) and step (d).

4. A method as claimed in claim 3 wherein the NCKX molecule is NCKX5.

5. A method as claimed in claim 4 further comprising the step of comparing the calcium ion movement in response to a test compound to a control measurement.

6. A method as claimed in claim 5 further comprising the steps of:
(f) repeating the above steps (a), (b), (d) and (e) to provide a control result for the change in the Calcium ion (Ca²⁺) concentration without exposure to one or more test compounds;
(g) comparing the amount of Calcium ion (Ca²⁺) movement across the membrane identified in step (e) after exposure to the test compound, and amount of Calcium ion (Ca²⁺) movement across the membrane in the control of step (f);
(h) identifying whether the amount of Calcium ion (Ca²⁺) movement across the membrane has increased, decreased or stayed the same in response to exposure to the test compound(s).

7. A method as claimed in claim 5 further comprising the step of comparing the amount of Calcium ion movement across a control membrane which does not contain a NCKX protein.

8. A method as claimed in claim 3 wherein an increase in the amount of Calcium ion (Ca²⁺) movement across the membrane indicates the test compound(s) increase skin and/or hair pigmentation and a decrease in the amount of Calcium ion (Ca²⁺) movement across the membrane indicates the test compound(s) decrease skin and/or hair pigmentation.

9. A method as claimed in claim 3 further comprising the step of:
(i) isolating the one or more test compounds.

10. A method as claimed in claim 9 further comprising the step of:
(j) formulating the one or more test compounds isolated in step (i) into a cosmetic or pharmaceutical formulation.

11. A method as claimed in claim 3 wherein the membrane is a biological membrane.

12. A method as claimed in claim 11 wherein the biological membrane is a cell membrane.

13. A method as claimed in claim 12 wherein the cell membrane is part of an intact cell.

14. A method as claimed in claim 13 wherein the cell membrane and/or intact cell is one selected from Hamster Embryonic Kidney (HEK) cells, High five insect cells, yeast cells, dictyostelium cells, tobacco plant cells, p53 deficient cell line H1299 and/or bacteria.

15. A method as claimed in claim 1 wherein the NCKX molecule is located in the membrane naturally, is artificially targeted to the membrane or is reconstituted in an artificial membrane.

16. A method as claimed in claim 15 wherein the NCKX is artificially targeted to the membrane by linking a leader sequence and/or tag that targets polypeptides to and for inclusion in a membrane.

17. A method as claimed in claim 16 wherein the leader sequence is derived from NCKX2 or 4, yeast a mating factor, NCX proteins, TGFbeta, haemagglutinin or viral surface proteins.

18. A method as claimed in claim 17 wherein the leader sequence is the N terminal sequence of hsNCKX2 (amino acids 1 to 120).

19. A method as claimed in claim 1 wherein the Calcium ion (Ca²⁺) concentration and/or movement is measured using a method selected from Ca²⁺ sensitive dyes (fluorescent and/or non-fluorescent), patch clamp, or radioactive calcium.

20. A method as claimed in claim 1 wherein the NCKX molecule or functionally equivalent variant, fusion or derivative thereof possesses a single nucleotide polymorphism (SNP) at the equivalent codon for amino acid residue 111.

21. A method as claimed in claim 20 wherein the SNP at the equivalent codon for amino acid residue 111 can be either Alanine or Threonine.

22. A nucleic acid molecule encoding a fusion protein comprising the nucleic acid molecule encoding a NCKX molecule or a functionally equivalent variant, fusion or derivative thereof and a nucleic acid molecule encoding a membrane targeting leader peptide and/or tag.

23. A nucleic acid molecule as claimed in claim 22 wherein the NCKX molecule is NCKX5.

24. A nucleic acid molecule as claimed in claim 23 wherein the membrane targeting leader peptide and/or tag is derived from NCKX2 or 4, yeast a mating factor, NCX proteins, TGFbeta, haemagglutinin or viral surface proteins.

25. A nucleic acid molecule as claimed in claim 24 wherein the leader sequence is the N terminal sequence of hsNCKX2 (amino acids 1 to 120).

26. A nucleic acid molecule claimed in claim 25 wherein the nucleic acid molecule encoding the NCKX molecule or a functionally equivalent variant, fusion or derivative thereof possesses a single nucleotide polymorphism (SNP) at the equivalent codon for amino acid residue 111.

27. A nucleic acid molecule as claimed in claim 26 wherein the SNP at the codon for amino acid residue 111 can be either Alanine or Threonine.

28. An expression vector comprising a nucleic acid molecule as claimed in claim 27.

29. A host cell containing a nucleic acid molecule and/or an expression vector as claimed in claim 22.

30. A host cell containing a nucleic acid molecule encoding a NCKX molecule or a functionally equivalent variant, fusion or derivative thereof and a nucleic acid molecule encoding a membrane targeting leader peptide and/or tag and/or expression vector comprising said nucleic acid molecule, said host cell further displaying at its surface the polypeptide encoded by the nucleic acid molecule and/or an expression vector.

31. A polypeptide comprising a polypeptide encoded by the nucleic acid molecule of claim 22.

32. A kit of parts comprising:
(i) at least one membrane including at least one polypeptide as defined in claim 31 and/or at least one cell displaying at its surface at least one polypeptide as defined in claim 31;
(ii) either a solid support to which the at least one membrane and/or the at least one cell may be fixed, or a solution which the at least one membrane and/or the at least one cell may be suspended;
(iii) a multi-welled plate;
(iv) a calcium sensitive detection system and
(v) instructions on using the kit.

33. A kit as claimed in claim 32 wherein the calcium detection system is a calcium sensitive dye.

34. A method for the treatment or prevention of disease characterised by excessive pigmentation and/or reduced pigmentation and/or in the prevention of sun-induced skin damage and/or skin cancer which comprises administering to a host in need of such treatment or prevention, an effective amount of isolate in step (i) of claim 9.

35. The method as claimed in claim 34 wherein compounds increasing calcium movement are used for treatment or prevention of disease characterised by reduced pigmentation and/or for the prevention of sun-induced skin damage and/or skin cancer and/or diseases characterised by vitamin D deficiency.

36. The method as claimed in claim 34 wherein compounds reducing calcium movement are used for treatment or prevention of disease characterised by elevated pigmentation.

37. A cosmetic product for increasing and/or reducing skin and/or hair pigmentation comprising a compound isolated in step (i) of claim 1.

38. A composition as claimed in claim 37 including a compound which increases calcium movement.

39. A composition as claimed in claim 37 including a compound which reduces calcium movement.

40.-47. (canceled)


SEQUENCE LISTING (61 sequences)

SEQ ID NO: 1 - protein, 11 aa, Artificial Sequence
  Synthetic SNP region
DVAGATFMAAG

SEQ ID NO: 2 - protein, 9 aa, Artificial Sequence
  Synthetic Myc tag
QKLISEEDL

SEQ ID NO: 3 - DNA, 1617 bp, Homo Sapiens
cctcacatgatccagtttaatcctcctcttctcccttcctgaagctgcacgctgcagtaagagcacagcagaaatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggagtttcccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcacaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaataactggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgatattattaatagtgttatgcagaaaatatgaatggcaggga

SEQ ID NO: 4 - protein, 500 aa, Homo Sapiens
MQTKGGQTWARRALLLGILWATAHLPLSGTSLPQRLPRATGNSTQCVISPSSEFPEGFFTRQERRDGGIIIYFLIIVYMFMAISIVCDEYFLPSLEIISESLGLSQDVAGTTFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 5 - protein, 46 aa, Homo Sapiens
IVCDDFFVPSLEKICERLHLSEDVAGATFMAAGSSTPELFASVIGV

SEQ ID NO: 6 - protein, 46 aa, Homo Sapiens
IVCDDFFVPSLEKICERLHLSEDVAGATFMAAGSSAPELFTSVIGV

SEQ ID NO: 7 - protein, 46 aa, Homo Sapiens
IVCDEYFLPSLEIISESLGLSQDVAGTTFMAAGSSAPELVTAFLGV

SEQ ID NO: 8 - protein, 46 aa, Homo Sapiens
IVCDEFFVPSLTVITEKLGISDDVAGATFMAAGGSAPELFTSLIGV

SEQ ID NO: 9 - protein, 46 aa, Homo Sapiens
IVCDEYFVPALGVITDKLQISEDVAGATFMAAGGSAPELFTSLIGV

SEQ ID NO: 10 - DNA, 300 bp, Artificial Sequence
  Synthetic pIE1/153A (V4-) polylinker
ctaattcaagaggtgcgacgaagaagttgccgcgttggtagtagacggtatcgataagcttgatatcgaattcctgcagccctgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttcgagaagtcttacgaacttcccgacggtcaggtcatcaccatcggaaacgaaag

SEQ ID NO: 11 - DNA, 300 bp, Artificial Sequence
  Synthetic pIE1/153A (V4+) polylinker
ctaattcaagaggtgcgacgaagaagttgccgcgttggtagtagacggtatcgataagcttgatatcgaattcctgcagcccaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattagacgagaagtcttacgaacttcccgacggtcaggtcatcaccatcggaaacgaaag

SEQ ID NO: 12 - DNA, 22 bp, Artificial Sequence
  Synthetic Actin 1 primer
gacgaagaagttgccgcgttgg

SEQ ID NO: 13 - DNA, 22 bp, Artificial Sequence
  Synthetic Actin 3 primer
cgatggtgatgacctgaccgtc

SEQ ID NO: 14 - DNA, 1547 bp, Artificial Sequence
  Synthetic myc tagged human nckx5
ctcgagatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggaacagaagctcatctcagaagaggatctggaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgagcggccgc

SEQ ID NO: 15 - protein, 510 aa, Artificial Sequence
  Synthetic myc tagged human nckx5
MQTKGGQTWARRALLLGILWATAHLPLSGTSLPQRLPRATGNSTQCVISPSSEQKLISEEDLEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 16 - DNA, 1577 bp, Artificial Sequence
  Synthetic myc and id4 tagged human nckx5
ctcgagatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggaacagaagctcatctcagaagaggatctggaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggtacagagacatcccaggtcgcaccagcatagtgagcggccgc

SEQ ID NO: 17 - protein, 519 aa, Artificial Sequence
  Synthetic myc and id4 tagged human nckx5
MQTKGGQTWARRALLLGILWATAHLPLSGTSLPQRLPRATGNSTQCVISPSSEQKLISEEDLEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGGTETSQVAPA

SEQ ID NO: 18 - DNA, 1751 bp, Artificial Sequence
  Synthetic myc tagged nckx2/nckx5 chimera
ctcgagatggatctgcaacaaagcaccaccatcacttccctagagaaatggtgtttggatgagtcactgtctggctgcagaagacattacagtgtcaagaaaaaactgaagttaattcgagtcttaggccttttcatgggtctggtagccattagcactgtctcattttcaatcagtgccttttctgagacagatacacagagcacaggagaggccagtgttgtaagtggccctagggtagcacagggttaccatgaacaaaagctgatcagcgaagaagatctgggttaccatcagagaactctcttagatttaaatgacaagattctggattatactccacagccacctctttctaaggaaggcgagtctgagaatagtacagatcacgcccaagaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgagcggccgc

SEQ ID NO: 19 - protein, 578 aa, Artificial Sequence
  Synthetic myc tagged nckx2/nckx5 chimera
MDLQQSTTITSLEKWCLDESLSGCRRHYSVKKKLKLIRVLGLFMGLVAISTVSFSISAFSETDTQSTGEASVVSGPRVAQGYHEQKLISEEDLGYHQRTLLDLNDKILDYTPQPPLSKEGESENSTDHAQEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 20 - DNA, 1781 bp, Artificial Sequence
  Synthetic myc tagged human nckx2/nckx5 chimera with id4 tag
ctcgagatggatctgcaacaaagcaccaccatcacttccctagagaaatggtgtttggatgagtcactgtctggctgcagaagacattacagtgtcaagaaaaaactgaagttaattcgagtcttaggccttttcatgggtctggtagccattagcactgtctcattttcaatcagtgccttttctgagacagatacacagagcacaggagaggccagtgttgtaagtggccctagggtagcacagggttaccatgaacaaaagctgatcagcgaagaagatctgggttaccatcagagaactctcttagatttaaatgacaagattctggattatactccacagccacctctttctaaggaaggcgagtctgagaatagtacagatcacgcccaagaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggtacagagacatcccaggtcgcaccagcatagtgagcggccgc

SEQ ID NO: 21 - protein, 587 aa, Artificial Sequence [sequence omitted]

SEQ ID NO: 22 - RNA, 25 nt, Artificial Sequence
  Synthetic RNAi duplex 370 antisense oligo
guccuacaacguccacguugaaagu

SEQ ID NO: 23 - RNA, 25 nt, Artificial Sequence
  Synthetic RNAi duplex 370 sense oligo
caggauguugcaggugcaacuuuca

SEQ ID NO: 24 - RNA, 25 nt, Artificial Sequence
  Synthetic RNAi duplex 370 scrambled control antisense oligo
gucaacacugcgcacaguuaucagu

SEQ ID NO: 25 - RNA, 25 nt, Artificial Sequence
  Synthetic RNAi duplex 370 scrambled control sense oligo
caguugugacgcgugucaauaguca

SEQ ID NO: 26 - RNA, 25 nt, Artificial Sequence
  Synthetic RNAi duplex 762 antisense oligo
gucucgauaccuccucucuuaacuu

SEQ ID NO: 27 - RNA, 25 nt, Artificial Sequence
  Synthetic RNAi duplex 762 sense oligo
cagagcuauggaggagagaauugaa

SEQ ID NO: 28 - RNA, 25 nt, Artificial Sequence
  Synthetic RNAi duplex 762 scrambled control antisense oligo
gucauagcuccucucauucacucuu

SEQ ID NO: 29 - RNA, 25 nt, Artificial Sequence
  Synthetic RNAi duplex 762 scrambled control sense oligo
caguaucgaggagaguaagugagaa

SEQ ID NO: 30 - RNA, 25 nt, Artificial Sequence
  Synthetic RNAi duplex 1265 antisense oligo
agcacccuagguuacacaaacuaua

SEQ ID NO: 31 - RNA, 25 nt, Artificial Sequence
  Synthetic RNAi duplex 1265 sense oligo
ucgugggauccaauguguuugauau

SEQ ID NO: 32 - RNA, 25 nt, Artificial Sequence
  Synthetic RNAi duplex 265 antisense oligo
ccauagcauuagaugaaggaguaau

SEQ ID NO: 33 - RNA, 25 nt, Artificial Sequence
  Synthetic RNAi duplex 265 sense oligo
gguaucguaaucuacuuccucauua

SEQ ID NO: 34 - RNA, 25 nt, Artificial Sequence
  Synthetic RNAi duplex 567 antisense oligo
ggagaaggcucugacacgucacaua

SEQ ID NO: 35 - RNA, 25 nt, Artificial Sequence
  Synthetic RNAi duplex 567 sense oligo
ccucuuccgagacugugcaguguau

SEQ ID NO: 36 - DNA, 18 bp, Artificial Sequence
  Synthetic SLC24A1 primer forward
tctgcacaacagcaccat

SEQ ID NO: 37 - DNA, 20 bp, Artificial Sequence
  Synthetic SLC24A1 primer reverse
ctctcctcctccttctcctt

SEQ ID NO: 38 - DNA, 19 bp, Artificial Sequence
  Synthetic SLC24A2 primer forward
atgatacacacccttgacc

SEQ ID NO: 39 - DNA, 21 bp, Artificial Sequence
  Synthetic SLC24A2 primer reverse
ccttttctctgaacctccctt

SEQ ID NO: 40 - DNA, 22 bp, Artificial Sequence
  Synthetic SLC24A3 primer forward
cgtcttatacttcactgtaccc

SEQ ID NO: 41 - DNA, 21 bp, Artificial Sequence
  Synthetic SLC24A3 primer reverse
aaccaatgattgtgaccatcc

SEQ ID NO: 42 - DNA, 18 bp, Artificial Sequence
  Synthetic SLC24A4 primer forward
gacacagacagccaagaa

SEQ ID NO: 43 - DNA, 24 bp, Artificial Sequence
  Synthetic SLC24A4 primer reverse
gcatagaacatatacagagcacca

SEQ ID NO: 44 - DNA, 22 bp, Artificial Sequence
  Synthetic SLC24A5 primer forward
gagatggaggcatcataatcta

SEQ ID NO: 45 - DNA, 22 bp, Artificial Sequence
  Synthetic SLC24A5 primer reverse
cctgagacaatccaagggattc

SEQ ID NO: 46 - DNA, 17 bp, Artificial Sequence
  Synthetic SLC24A6 primer forward
aggcttcactggctctt

SEQ ID NO: 47 - DNA, 20 bp, Artificial Sequence
  Synthetic SLC24A6 primer reverse
aggcatctccaatgctgttc

SEQ ID NO: 48 - DNA, 18 bp, Artificial Sequence
  Synthetic GAPDH primer forward
ggacctgacctgccgtct

SEQ ID NO: 49 - DNA, 18 bp, Artificial Sequence
  Synthetic GAPDH primer reverse
tagcccaggatgcccttg

SEQ ID NO: 50 - protein, 10 aa, Artificial Sequence
  Synthetic NCKX5 C-terminal peptide
GNNKIRGCGG

SEQ ID NO: 51 - DNA, 25 bp, Artificial Sequence
  Synthetic RNAi duplex 185 sense oligo
agggccacaggaaatagcacccaat

SEQ ID NO: 52 - DNA, 25 bp, Artificial Sequence
  Synthetic RNAi duplex 185 antisense oligo
attgggtgctatttcctgtggccct

SEQ ID NO: 53 - DNA, 25 bp, Artificial Sequence
  Synthetic RNAi duplex 260 sense oligo
gagcgcagagatggaggcatcataa

SEQ ID NO: 54 - DNA, 25 bp, Artificial Sequence
  Synthetic RNAi duplex 260 antisense oligo
ttatgatgcctccatctctgcgctc

SEQ ID NO: 55 - DNA, 25 bp, Artificial Sequence
  Synthetic RNAi duplex 301 sense oligo
cgtttacatgttcatggccatatct

SEQ ID NO: 56 - DNA, 25 bp, Artificial Sequence
  Synthetic RNAi duplex 301 antisense oligo
agatatggccatgaacatgtaaacg

SEQ ID NO: 57 - DNA, 25 bp, Artificial Sequence
  Synthetic RNAi duplex 492 sense oligo
gcaccatccttggatctgcaattta

SEQ ID NO: 58 - DNA, 25 bp, Artificial Sequence
  Synthetic RNAi duplex 492 antisense oligo
taaattgcagatccaaggatggtgc

SEQ ID NO: 59 - DNA, 25 bp, Artificial Sequence
  Synthetic RNAi duplex 1110 sense oligo
ccgcatttacatatatcctggtttg

SEQ ID NO: 60 - DNA, 25 bp, Artificial Sequence
  Synthetic RNAi duplex 1110 antisense oligo
caaaccaggatatatgtaaatgcgg

SEQ ID NO: 61 - protein, 16 aa, Artificial Sequence
  Synthetic peptide sequence for NCKX5 cytosolic loop
DEGQPFIRRQSRTDSG